(19) United States
(12) Patent Application Publication
(10) Pub. No.: US 2025/0170366 A1
(43) Pub. Date: May 29, 2025

(54) PULL WIRE RETAINER FOR STEERABLE MEDICAL DEVICE

(71) Applicant: Boston Scientific Scimed, Inc., Maple Grove, MN (US)

(72) Inventor: Jeffery D. Arnett, Louisa, VA (US)

(21) Appl. No.: 18/963,025

(22) Filed: Nov. 27, 2024

Related U.S. Application Data
(60) Provisional application No. 63/602,960, filed on Nov. 27, 2023.
(60) Provisional application No. 63/616,429, filed on Dec. 29, 2023.

(51) Int. Cl. A61M 25/01

(52) U.S. Cl. CPC A61M 25/0136; A61M 25/0147

(57) ABSTRACT

A handle for a steerable medical device having a first control wire and a second control wire, a distal end of each of the control wires being coupled to the medical device at a distal region thereof includes a housing. A slide assembly is positioned within the housing and operable to translate linearly therein. The slide assembly includes a first wire retainer and a second wire retainer. A knob is rotatably coupled to the housing, for linearly translating the slide assembly, thereby enabling the slide assembly to manipulate the first control wire and the second control wire to effect a change in a deflection of the medical device. The slide assembly comprises a ring-shape formed from a first section, a second section, and a third section. The first section and the third section are separated by the second section.

### CROSS REFERENCE TO RELATED APPLICATIONS

This application claims priority to U.S. Provisional Patent Application No. 63/602,960 entitled “PULL WIRE RETAINER FOR STEERABLE MEDICAL DEVICE,” filed Nov. 27, 2023, and U.S. Provisional Patent Application No. 63/616,429 entitled “PULL WIRE RETAINER FOR STEERABLE MEDICAL DEVICE,” filed Dec. 29, 2023, which are hereby incorporated by reference in their entirety.

### TECHNICAL FIELD

The disclosure relates to a handle for a medical device. More specifically the disclosure relates to a wire retainer for use with a handle for a medical device that enables steering of the medical device in the body of a patient.

### BACKGROUND

U.S. Pat. No. 5,944,690 granted to Falwell et al. discloses a steerable catheter control mechanism for manipulating a pair of control wires which utilizes a slider mechanism coupled to the proximal ends of the control wires. However, the slider mechanism disclosed by Falwell lacks ease of use as it is awkward to grasp and use. Furthermore, the disclosed slider mechanism provides limited control in steering the catheter. The device provides a thumb control that lacks precision. It is unable to provide precise steering of the catheter as it lacks resolution for permitting minute manipulations needed to provide slight changes in the deflection of the catheter.

U.S. Pat. No. 7,691,095 granted to Bednarek et al. discloses a bi-directional steerable catheter control handle which includes an adjustment knob rotatably connected to the handle. Rotation of the handle results in deflection of two sliding members (each connected to a pull wire) in opposite directions, resulting in respective deflection of the distal end of the catheter. However, the steerable control handle provided by Bednarek is complex and difficult to manufacture.

### SUMMARY

Example 1 is a handle for a steerable medical device. The medical device includes a first control wire, a distal end of the first control wire being coupled to the medical device at a distal region thereof. The handle includes a housing, and a slide assembly positioned within the housing operable to translate linearly therein. The slide assembly comprises a first wire retainer. A knob is rotatably coupled to the housing, for linearly translating the slide assembly, thereby enabling the slide assembly to manipulate the first control wire to effect a change in a deflection of the medical device. The slide assembly comprises a ring-shape formed from a first section, a second section, and a third section. The first section and the third section are separated by the second section.

Example 2 is the handle of Example 1, wherein the medical device includes a second control wire, a distal end of the second control wire being coupled to the medical device at a distal region thereof; and wherein the knob is configured for linearly translating the slide assembly, thereby enabling the slide assembly to manipulate the second control wire to effect a change in a deflection of the medical device.

Example 3 is the handle of Examples 1 or 2, wherein first section and the third section form a first half of the ring-shape and the second section forms a second half of the ring-shape.

Example 4 is the handle of any one of Examples 1-3, further comprising a screw that includes a thread, wherein rotation of the knob causes rotation of the thread.

Example 5 is the handle of Example 4, wherein the first section, the second section, and the third section comprise a threaded inner surface that corresponds to the thread of the screw.

Example 6 is the handle of any one of Examples 1-5, wherein the first wire retainer includes a distal face having a channel, a proximal face having channel, and a first wire retainer block located between the distal face and the proximal face.

Example 7 is the handle of Example 6, further comprising a second wire retainer, wherein the second wire retainer includes a distal face having a channel, a proximal face having channel, and a second wire retainer block located between the distal face and the proximal face.

Example 8 is the handle of Example 7, wherein the first wire retainer block has a proximal end, a distal end, and an overhang extending between the first wire retainer block proximal end and the first wire retainer block distal end, and the second wire retainer block has a proximal end, a distal end, and an overhang extending between the second wire retainer block proximal end and the second wire retainer block distal end.

Example 9 is the handle of any of Examples 1-8, wherein the first wire retainer partially extends across an outer surface of the first section, the second section, and the third section.

Example 10 is the handle of any of Examples 1-5, wherein the first wire retainer includes a hypotube.

Example 11 is the handle of Example 4, wherein the knob is integral with

the screw.

Example 12 is the handle of any of Examples 1-10, wherein the knob is located at a proximal end of the housing.

Example 13 is the handle of any of Examples 1-13, wherein the first section includes an outer surface having a first raised portion configured to translate within a first guide that extends along an inner portion of the housing.

Example 14 is the handle of Example 13, wherein the second section includes an outer surface having a second raised portion configured to translate within a second guide that extends along an inner portion of the housing.

Example 15 is the handle of Example 14, wherein the first raised portion and the first guide are positioned off center from a longitudinal axis passing through the handle.

Example 16 is a handle for a steerable medical device, the medical device including a first control wire and a second control wire, a distal end of each of the control wires being coupled to the medical device at a distal region thereof. The handle includes a housing. A slide assembly is positioned within the housing and operable to translate linearly therein. The slide assembly includes a first wire retainer and a second wire retainer. A knob is rotatably coupled to the housing, for linearly translating the slide assembly, thereby enabling the slide assembly to manipulate the first control wire and the second control wire to effect a change in a deflection of the medical device. The slide assembly comprises a ring-shape formed from a first section, a second section, and a third section. The first section and the third section are separated by the second section.

Example 17 is the handle of Example 16, wherein first section and the third section form a first half of the ring-shape and the second section forms a second half of the ring-shape.

Example 18 is the handle of Example 16, further comprising a screw that includes a thread, wherein rotation of the knob causes rotation of the thread.

Example 19 is the handle of Example 18, wherein the first section, the second section, and the third section comprise a threaded inner surface that corresponds to the thread of the screw.

Example 20 is the handle of Example 16, wherein the first wire retainer includes a distal face having a channel, a proximal face having channel, and a first wire retainer block located between the distal face and the proximal face.

Example 21 is the handle of Example 20, further comprising a second wire retainer, wherein the second wire retainer includes a distal face having a channel, a proximal face having channel, and a second wire retainer block located between the distal face and the proximal face.

Example 22 is the handle of Example 21, wherein the first wire retainer block has a proximal end, a distal end, and an overhang extending between the first wire retainer block proximal end and the first wire retainer block distal end, and the second wire retainer block has a proximal end, a distal end, and an overhang extending between the second wire retainer block proximal end and the second wire retainer block distal end.

Example 23 is the handle of Example 16, wherein the first wire retainer partially extends across an outer surface of the first section, the second section, and the third section.

Example 24 is the handle of Example 16, wherein the first wire retainer includes a hypotube.

Example 25 is the handle of Example 18, wherein the knob is integral with the screw.

Example 26 is the handle of Example 16, wherein the knob is located at a proximal end of the housing.

Example 27 is the handle of Example 16, wherein the first section includes an outer surface having a first raised portion configured to translate within a first guide that extends along an inner portion of the housing.

Example 28 is the handle of Example 27, wherein the second section includes an outer surface having a second raised portion configured to translate within a second guide that extends along an inner portion of the housing.

Example 29 is the handle of Example 28, wherein the first raised portion and the first guide are positioned off center from a longitudinal axis passing through the handle.

Example 30 is a slide assembly for a steerable medical device, the medical device including at least one control wire configured to impart a change in shape of the steerable medical device. The slide assembly includes a first section having a threaded inner surface and an outer surface, the outer surface including a first raised portion. The slide assembly includes a second section having a threaded inner surface and an outer surface, the outer surface including a second raised portion. A first wire retainer extends partially along the first section outer surface and the second section outer surface. A second wire retainer extends partially along the first section outer surface and the second section outer surface.

Example 31 is the slide assembly of Example 30, wherein the first wire retainer is located on an opposite side of the outer surface than the second wire retainer.

Example 32 is the slide assembly of Example 30, further comprising a third section, wherein the first section and the third section are separated by the second section.

Example 33 is the slide assembly of Example 32, wherein the first section, the second section, and the third section form a generally ring-shape.

Example 34 is a slide assembly for a steerable medical device, the medical device including at least one control wire configured to impart a change in shape of the steerable medical device. The slide assembly includes a first section having a threaded inner surface and an outer surface. The slide assembly includes a second section having a threaded inner surface and an outer surface. The slide assembly includes a third section having a threaded inner surface and an outer surface. A first wire retainer extends partially along the first section outer surface, the second section outer surface, and the third section outer surface. The first section, the second section, and the third section form a generally ring-shape.

Example 35 is the slide assembly of Example 34, further comprising a second wire retainer extending partially along the first section outer surface, the second section outer surface, and the third section outer surface.

Example 36 is a handle for a steerable medical device, the medical device including a first control wire, a distal end of the first control wire being coupled to the medical device at a distal region thereof. The handle includes a housing. A slide assembly is positioned within the housing and is operable to translate linearly therein. The slide assembly includes a nut and a wire retainer. A knob is rotatably coupled to the housing, for linearly translating the slide assembly, thereby enabling the slide assembly to manipulate the first control wire to effect a change in a deflection of said medical device. The wire retainer includes a distal face having a first channel and a second channel, a proximal face having a first channel and a second channel, a first block located between the distal face and the proximal face, and a second block located between the distal face and the proximal face.

Example 37 is the handle of Example 36, wherein the medical device includes a second control wire, a distal end of the second control wire being coupled to the medical device at a distal region thereof; and wherein the knob is configured for linearly translating the slide assembly, thereby enabling the slide assembly to manipulate the second control wire to effect a change in a deflection of the medical device.

Example 38 is the handle of Example 36 or 37, wherein the first block has a proximal end, a distal end, and an overhang extending between the first block proximal end and the first block distal end, and the second block has a proximal end, a distal end, and an overhang extending between the second block proximal end and the second block distal end.

Example 39 is the handle of Example 38, wherein the first block and the second block extend partially between the distal face and the proximal face such that gaps are located adjacent the first block proximal end, the first block distal end, the second block proximal end, and the second block distal end.

Example 40 is the handle of any one of Examples 36-39, wherein the wire retainer includes a curved outer surface that extends between the distal face and the proximal face.

Example 41 is the handle of any of Examples 36-40, wherein the distal face first channel, first block, and proximal face first channel are configured to retain the first control wire and the distal face second channel, second block, and proximal face second channel are configured to retain the second control wire.

Example 42 is the handle of any one of Examples 36-41, wherein the slide assembly comprises a bridge connecting the nut and the wire retainer.

Example 43 is the handle of Example 42, wherein the bridge includes a curved surface.

Example 44 is the handle of any one of Examples 36-43, further comprising a screw that includes a thread, wherein rotation of the knob causes rotation of the thread.

Example 45 is the handle of Example 44, wherein the knob is integral with the screw.

Example 46 is the handle of any of Examples 44 or 45, wherein the knob includes an internal knob and an external knob connected together, wherein the internal knob connects to a head of the screw.

Example 47 is the handle of any of Examples 36-46, wherein the knob is located at a distal end of the housing.

Example 48 is the handle of any of Examples 36-45, wherein the knob is located at a proximal end of the housing.

Example 49 is the handle of any of Examples 36-48, wherein the nut includes a first raised portion configured to translate within a first guide that extends along an inner portion of the housing.

Example 50 is the handle of any of Examples 43-48, wherein the slide assembly includes an extension that fits within a channel that extends along an inner portion of the housing.

Example 51 is a handle for a steerable medical device, the medical device including a first control wire and a second control wire, a distal end of each of the control wires being coupled to the medical device at a distal region thereof. The handle includes a housing. A slide assembly is positioned within the housing and is operable to translate linearly therein. The slide assembly includes a nut, a bridge, and a wire retainer. A knob is rotatably coupled to the housing, for linearly translating the slide assembly, thereby enabling the slide assembly to separately manipulate each of said first control wire and said second control wire to effect a change in a deflection of said medical device. The wire retainer includes a distal face having a first channel and a second channel, a proximal face having a first channel and a second channel, a first block located between the distal face and the proximal face, and a second block located between the distal face and the proximal face.

Example 52 is the handle of Example 51, wherein the first block has a proximal end, a distal end, and an overhang extending between the first block proximal end and the first block distal end.

Example 53 is the handle of Example 51, wherein the second block has a proximal end, a distal end, and an overhang extending between the second block proximal end and the second block distal end.

Example 54 is the handle of Example 51, wherein the first block and the second block extend partially between the distal face and the proximal face such that gaps are located adjacent the first block proximal end, the first block distal end, the second block proximal end, and the second block distal end.

Example 55 is the handle of Example 51, wherein the wire retainer includes a curved outer surface that extends between the distal face and the proximal face.

Example 56 is the handle of Example 51, wherein the distal face first channel, first block, and proximal face first channel are configured to retain the first control wire and the distal face second channel, second block, and proximal face second channel are configured to retain the second control wire.

Example 57 is the handle of Example 51, wherein the bridge extends between the nut and the wire retainer.

Example 58 is the handle of Example 57, wherein the bridge includes a curved surface.

Example 59 is the handle of Example 51, further comprising a screw that includes a thread, wherein rotation of the knob causes rotation of the thread.

Example 60 is the handle of Example 59, wherein the knob is integral with the screw.

Example 61 is the handle of Example 59, wherein the knob includes an internal knob and an external knob connected together, wherein the internal knob connects to a head of the screw.

Example 62 is the handle of Example 51, wherein the knob is located at a distal end of the housing.

Example 63 is the handle of Example 51, wherein the knob is located at a proximal end of the housing.

Example 64 is the handle of Example 51, wherein the nut includes a first raised portion configured to translate within a first guide that extends along an inner portion of the housing.

Example 65 is the handle of Example 51, wherein the slide assembly includes an extension that fits within a channel that extends along an inner portion of the housing.

Example 66 is a slide assembly for a medical device. The slide assembly includes a nut having an internal thread, a wire retainer, and a bridge extending between the nut and the wire retainer. The wire retainer includes a distal face having a first channel and a second channel, a proximal face having a first channel and a second channel, a first block located between the distal face and the proximal face, and a second block located between the distal face and the proximal face.

Example 67 is the slide assembly of Example 66, wherein the first block has a proximal end, a distal end, and an overhang extending between the first block proximal end and the first block distal end, and the second block has a proximal end, a distal end, and an overhang extending between the second block proximal end and the second block distal end.

Example 68 is the slide assembly of Example 66, wherein the first block and the second block extend partially between the distal face and the proximal face such that gaps are located adjacent the first block proximal end, the first block distal end, the second block proximal end, and the second block distal end.

Example 69 is the slide assembly of Example 66, wherein the distal face first channel, first block, and proximal face first channel are configured to retain the first control wire and the distal face second channel, second block, and proximal face second channel are configured to retain the second control wire.

Example 70 is a wire retainer for a medical device. The wire retainer includes a distal face having a first channel and a second channel. The wire retainer includes a proximal face having a first channel and a second channel. A first block is located between the distal face and the proximal face. The first block includes a proximal end, a distal end, and an overhang extending between the first block proximal end and the first block distal end. A second block is located between the distal face and the proximal face. The second block includes a proximal end, a distal end, and an overhang extending between the second block proximal end and the second block distal end. The first block and the second block extend partially between the distal face and the proximal face such that gaps are located adjacent the first block proximal end, the first block distal end, the second block proximal end, and the second block distal end.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a side view of a handle assembly, in accordance with an embodiment of the present disclosure;

FIG. 2 is an exploded view of the handle assembly of FIG. 1, in accordance with an embodiment of the present disclosure;

FIG. 3A is a transverse cross-sectional view of the steerable handle of FIG. 1 showing a slide assembly in a most proximal position, in accordance with an embodiment of the present disclosure;

FIG. 3B is a transverse cross-sectional view of the steerable handle of FIG. 1 showing the slide assembly in a most distal position, in accordance with an embodiment of the present disclosure;

FIG. 3C is a cross-sectional view along line 3C in FIG. 3A, in accordance with an embodiment of the present disclosure;

FIG. 3D is a cross-sectional view along line 3D in FIG. 3A, in accordance with an embodiment of the present disclosure;

FIG. 4A is a perspective view of a wire retainer of the handle assembly of FIG. 1, in accordance with an embodiment of the present disclosure;

FIG. 4B is a cross-sectional view along line 4B in FIG. 4A, in accordance with an embodiment of the present disclosure;

FIG. 4C illustrates loading control wires into the wire retainer of FIG. 4A, in accordance with an embodiment of the present disclosure;

FIG. 5 is a side view of a handle assembly, in accordance with an embodiment of the present disclosure;

FIG. 6 is an exploded view of the handle assembly of FIG. 5, in accordance with an embodiment of the present disclosure;

FIG. 7A is a transverse cross-sectional view of the steerable handle of FIG. 5 showing a slide assembly in a most proximal position, in accordance with an embodiment of the present disclosure;

FIG. 7B is a transverse cross-sectional view of the steerable handle of FIG. 5 showing the slide assembly in a most distal position, in accordance with an embodiment of the present disclosure;

FIG. 7C is a cross-sectional view along line 7C in FIG. 7B, in accordance with an embodiment of the present disclosure;

FIG. 8 is a perspective view of a wire retainer of the handle assembly of FIG. 5, in accordance with an embodiment of the present disclosure.

FIG. 9 is a side view of a handle assembly, in accordance with an embodiment of the present disclosure;

FIG. 10 is an exploded view of the handle assembly of FIG. 9, in accordance with an embodiment of the present disclosure;

FIG. 11 is a perspective view of a wire retainer of the handle assembly of FIG. 9, in accordance with an embodiment of the present disclosure;

FIG. 12 is a perspective view of a handle assembly, in accordance with an embodiment of the present disclosure;

FIG. 13 is an exploded view of the handle assembly of FIG. 12, in accordance with an embodiment of the present disclosure;

FIG. 14A is a transverse partial cross-sectional view of the steerable handle of FIG. 12 showing a slide assembly in a most proximal position, in accordance with an embodiment of the present disclosure;

FIG. 14B is a transverse cross-sectional view of the steerable handle of FIG. 12 showing the slide assembly in a most distal position, in accordance with an embodiment of the present disclosure;

FIG. 15 is a cross-sectional view along line 15-15 in FIG. 14B, in accordance with an embodiment of the present disclosure;

FIG. 16A is a perspective view of an inside surface of a right handle housing of the handle assembly of FIG. 12, in accordance with an embodiment of the present disclosure;

FIG. 16B is a perspective view of an inside surface of a left handle housing of the handle assembly of FIG. 12, in accordance with an embodiment of the present disclosure;

FIG. 17 is a perspective view of a slide assembly of the handle assembly of FIG. 12, in accordance with an embodiment of the present disclosure;

FIG. 18 is a perspective view of a handle assembly, in accordance with an embodiment of the present disclosure;

FIG. 19 is an exploded view of the handle assembly of FIG. 18, in accordance with an embodiment of the present disclosure;

FIG. 20A is a transverse partial cross-sectional view of the steerable handle of FIG. 18 showing a slide assembly in a most proximal position, in accordance with an embodiment of the present disclosure;

FIG. 20B is a transverse cross-sectional view of the steerable handle of FIG. 18 showing the slide assembly in a most distal position, in accordance with an embodiment of the present disclosure;

FIG. 21 is a cross-sectional view along line 21-21 in FIG. 20B, in accordance with an embodiment of the present disclosure;

FIG. 22A is a perspective view of an inside surface of a right handle housing of the handle assembly of FIG. 18, in accordance with an embodiment of the present disclosure;

FIG. 22B is a perspective view of an inside surface of a left handle housing of the handle assembly of FIG. 18, in accordance with an embodiment of the present disclosure;

FIG. 22C is a transverse partial cross-sectional view of the steerable handle of FIG. 18 showing the location of ribs of the left handle housing in relation to the right handle housing, in accordance with an embodiment of the present disclosure;

FIG. 22D is a partial cutaway side view showing interaction of a first guide rib of the left handle housing with a control wire; in accordance with the present disclosure;

FIG. 23 is a perspective view of a slide assembly of the handle assembly of FIG. 18, in accordance with an embodiment of the present disclosure; and

FIG. 24 is a perspective view of a wire retainer of the handle assembly of FIG. 18, in accordance with an embodiment of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Steerable medical devices have various uses and applications, such as for guiding and positioning devices such as catheters, guidewires and the like within a patient's body. Handles used with such steerable devices typically include a mechanism for actuating one or more pull wires capable of deflecting the steerable device and thus steering or guiding a functional tip of a medical device positioned therein.

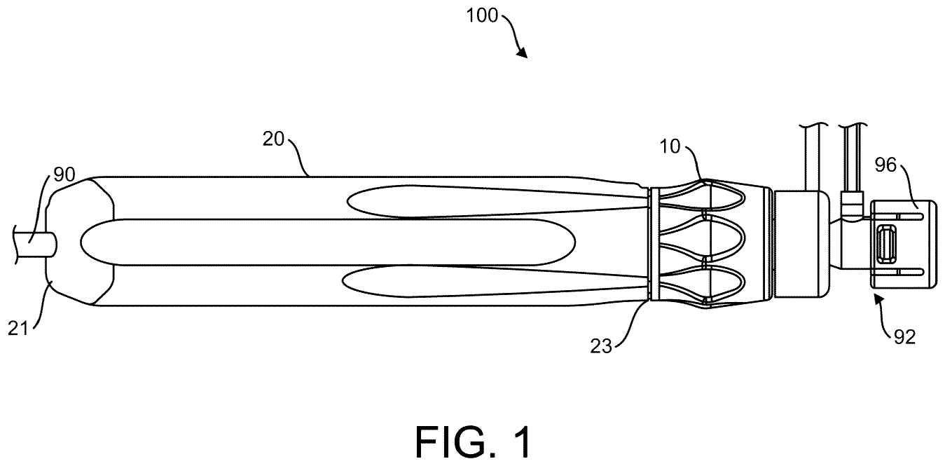

In one embodiment of the present disclosure as shown in FIG. 1, a steerable control system or handle 100 is provided for manipulating a medical device. The medical device may include, without limitation, a catheter, sheath, dilator, introducer, or similar medical devices. In a specific example, as shown, the handle 100 is coupled to a sheath 90 to enable a user to manipulate or steer the sheath 90 in a desired direction during use. The sheath 90 passes through a handle housing 20 from a proximal assembly 92 and extends distally from a distal end 21 of a handle housing 20.

The proximal assembly 92 includes a hub 96 having an aperture 98 that opens to a lumen that extends through the sheath 90. In one aspect, the aperture 98 is configured to receive an elongated medical device that can be inserted through the sheath 90, for example a guidewire, dilator, stylet, or other elongated medical device. In another aspect, the aperture 98 is configured to receive fluids, dyes, or medicaments that can be delivered to a desired location within a patient through the sheath 90.

The proximal assembly 92 includes an annular shoulder 94. The annular shoulder 94 is configured to be held by a recess 48 formed by a right hub holder 42 and a left hub holder 44 when assembled. The right hub holder 42 and the left hub holder 44 are configured to connect together using connecting elements 46, such as snap fit features to form a hub holder 43.

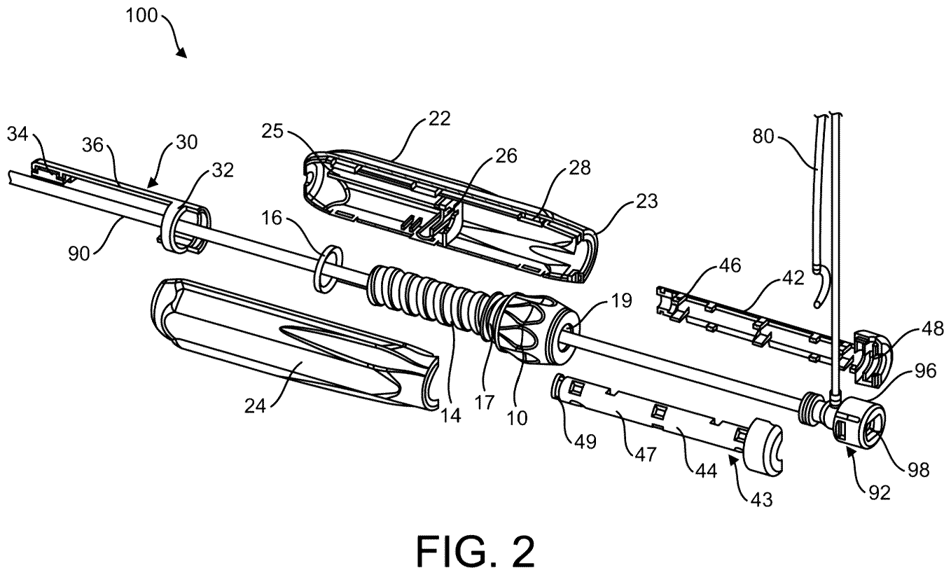

As shown in FIG. 2, the handle housing 20 includes a right handle housing 22 and a left handle housing 24 which are configured to join together to encase components within the handle housing 20. The right handle housing 22 and the left handle housing 24 include a hub holder support 26 configured to retain a distal end of the hub holder 43. The hub holder support 26 fits within an annular recess 49 at the distal end of hub holder 43. The hub holder support 26 ensures the longitudinal axis of a screw 14 that is supported by hub holder 43 remains centered along the longitudinal axis of the handle 100. The right handle housing 22 and the left handle housing 24 also include connecting elements 28, such as snap fit features, to allow the right handle housing 22 and the left handle housing 24 to be joined together.

The handle 100 comprises a knob 10 that is rotatably coupled to a proximal end 23 of the handle housing 20. The knob 10 is rotatable about the longitudinal axis of the handle 100 and rotates with respect to housing 20. In operation, the rotation of the knob 10 in a first rotational direction allows the user to steer or deflect the sheath 90 in a first direction, whereas the rotation of the knob 10 in a second rotational direction allows the user to steer or deflect the sheath 90 in a second direction.

The rotation of the knob 10 is converted into a deflection of the sheath 90 via a slide assembly 30 and screw 14, shown in FIG. 2. Generally, knob 10 includes an integrally molded screw 14 that rotates as knob 10 is rotated. In one embodiment, knob 10 can be formed of a softer material that is overmolded onto a portion of the screw 14 that extends out of housing 20. The knob 10 may include a plurality of raised portions 12 that aid a user in gripping the knob 10. An O-ring 16 sits in a channel 17 on the screw 14 and interacts with a projection 18 extending from an internal surface of the housing 20. The O-ring 16 is configured to dampen vibrations and provide a smooth feel as the knob 10 is rotated.

The knob 10 and screw 14 include a lumen 19 that passes through the longitudinal axis thereof. The hub holder 43 includes an elongate cylindrical body 47 that is configured to pass through the lumen 19 and support the knob 10 and screw 14. Sheath 90 passes through an interior lumen of the hub holder 43.

The screw 14 is co-operatively engaged with the slide assembly 30 which is housed within a lumen defined by the handle housing 20. The screw 14 includes a self-locking thread that communicates with a nut 32 of the slide assembly 30. As the knob 10 is rotated, the threads of the screw 14 cause translation of the slide assembly 30 via interaction with internal threads of the nut 32. In one embodiment, the slide assembly 30 translates proximally as the knob 10 is rotated in clockwise direction and distally when the knob 10 is rotated in a counter-clockwise direction. In another embodiment, the slide assembly 30 translates distally as the knob 10 is rotated in clockwise direction and proximally when the knob 10 is rotated in a counter-clockwise direction.

The slide assembly 30 includes the nut 32 at the proximal end, a wire retainer 34 at the distal end, and a bridge 36 joining the nut 32 and the wire retainer 34. The rotation of knob 10 causes a corresponding linear translation of the slide assembly 30 within the housing 20. This translation of the slide assembly 30 is converted into a tensioning of the control wires coupled to the slide assembly 30 and thereby resulting in a deflection of the sheath 90.

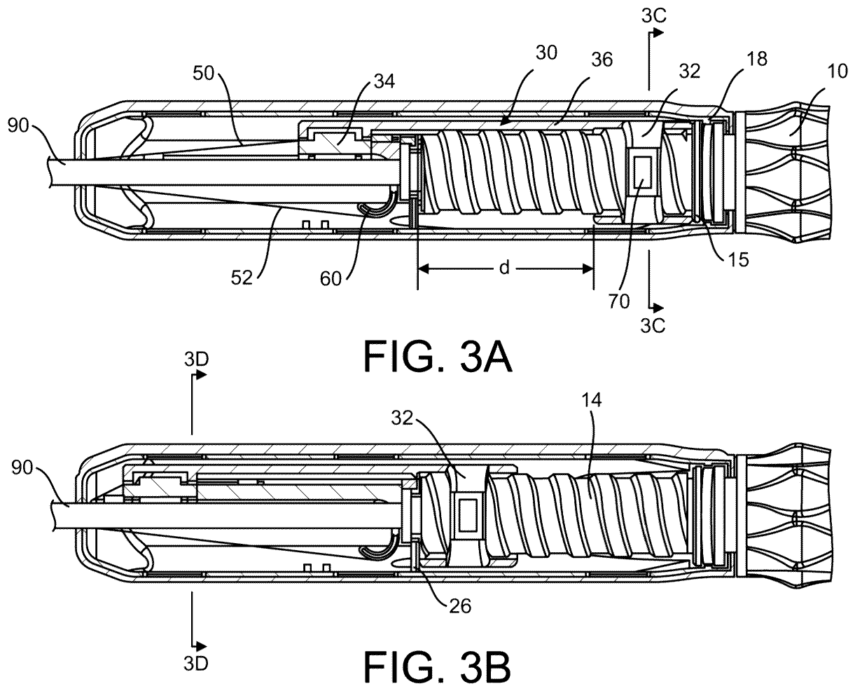

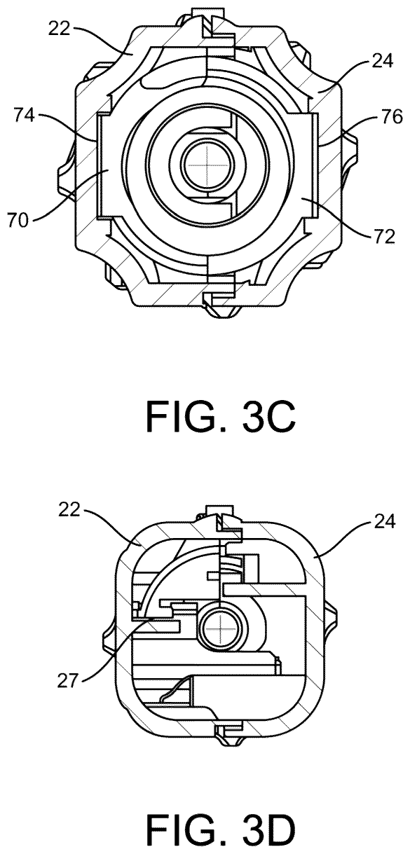

More specifically, slide assembly 30 is coupled to respective proximal ends of a pair of control wires that extend substantially along the length of the sheath 90, for example a first control wire 50 and a second control wire 52 as shown in FIG. 3A. A distal end (not shown) of each of the control wires 50, 52 is coupled to a distal portion of the sheath 90. The rotation of the knob 10 in one direction causes the slide assembly 30 to translate proximally within the housing 20 pulling one of the control wires (such as first control wire 50) to deflect the sheath 90 in a first direction, whereas the rotation of the knob 10 in an opposing direction causes the slide assembly 30 to translate distally within the housing 20 pulling the other of the control wires (such as second control wire 52) to deflect the sheath 90 in a second direction.

The amount of curvature imparted on the sheath 90 is limited by how far the slide assembly 30 translates within the handle 100. FIG. 3A shows the slide assembly 30 in a most proximal position. The slide assembly 30 is limited proximally by interaction of the nut 32 with flange 15 forming a portion of channel 17 to hold O-ring 16. FIG. 3B shows the slide assembly 30 in a most distal position. The slide assembly 30 is limited distally by interaction of the nut 32 with hub holder support 26. In one embodiment, the slide assembly 30 is configured to translate a within the handle 100 a distance d in the range of 1 to 2 inches. In one embodiment, the slide assembly 30 is configured to translate a distance d in the range of 1 to 1.5 inches. In another embodiment, the slide assembly 30 is configured to translate a distance d 1.38 inches.

FIG. 3C is a cross-sectional view through the nut 32 of slide assembly 30 along line 3C in FIG. 3A. As shown, the nut 32 includes a first raised portion 70 and a second raised portion 72 that is configured to translate within a first guide 74 and a second guide 76 respectively. The first guide 74 and the second guide 76 extend along the inner portion of right handle housing 22 and the left handle housing 24. The first guide 74 and the second guide 76 have a shape that corresponds to the shape of the first raised portion 70 and the second raised portion 72. In one embodiment, the first raised portion 70 and the second raised portion 72 are substantially rectangular as shown FIGS. 3A and 3B. The interaction of the raised portions 70, 72 and the guides 74, 76 allows the slide assembly 30 to translate along the handle 100 without rotating wile knob 10 is rotated.

FIG. 3D is a cross-sectional view through the wire retainer 34 of slide assembly 30 along line 3D in FIG. 3A. The right handle housing 22 includes a rib 25 that extends from hub holder support 26 to the distal end 21. As shown in FIG. 3D, the wire retainer 34 includes a surface 27 configured to interact with the rib 25. As such, the wire retainer 34 is supported and guided by rib 25 as the slide assembly 30 translates along the handle 100.

In one example as shown in FIG. 2, in order to allow the slide assembly 30 to separately impart a pulling force on each of the two control wires, one of the two control wires (such as first control wire 50) is directly coupled to the wire retainer 34 of slide assembly 30 whereas the other of the control wires (such as second control wire 52) is indirectly coupled to the slide assembly 30 via a direction reversing element 60 such a pulley or a pin. In some embodiments, the reversing element 60 may include a post, pin, curved guide, wall, or channel through which the second control wire 52 passes. In other words, a means for of coupling the distal ends of the wires 50, 52 to opposite sides of the sheath 90 is included in the handle, whereby motion of the slide assembly 30 in one direction will apply tension to one wire while motion of the slide in the other direction will apply tension to the other wire. As used herein, “directly coupled” is taken to mean that the proximal end of the wire is operably coupled (but not necessarily physically attached or integral with) to the slide assembly 30 without passing through an intermediate structure, while “indirectly coupled” is taken to mean that the proximal end of the wire is operably coupled (but not necessarily physically attached or integral with) to the slide assembly 30 after passing through an intermediate structure or element, such as a direction reversing element 60.

In one aspect, a proximal end of first control wire 50 exits sheath 90 and is coupled at or to the slide assembly 30 at the wire retainer 34. Thus, in this example, first control wire 50 is directly coupled to slide assembly 30 via the wire retainer 34. Similarly, a proximal end of second control wire 52 exits sheath 90 and is routed proximally. The second control wire 52 is then passed around or through the direction reversing element 60 and routed back distally so that it can be passed distally through the slide assembly 30 to be coupled at or to the wire retainer 34. Thus, in this example, second control wire 52 is indirectly coupled to the slide assembly 30.

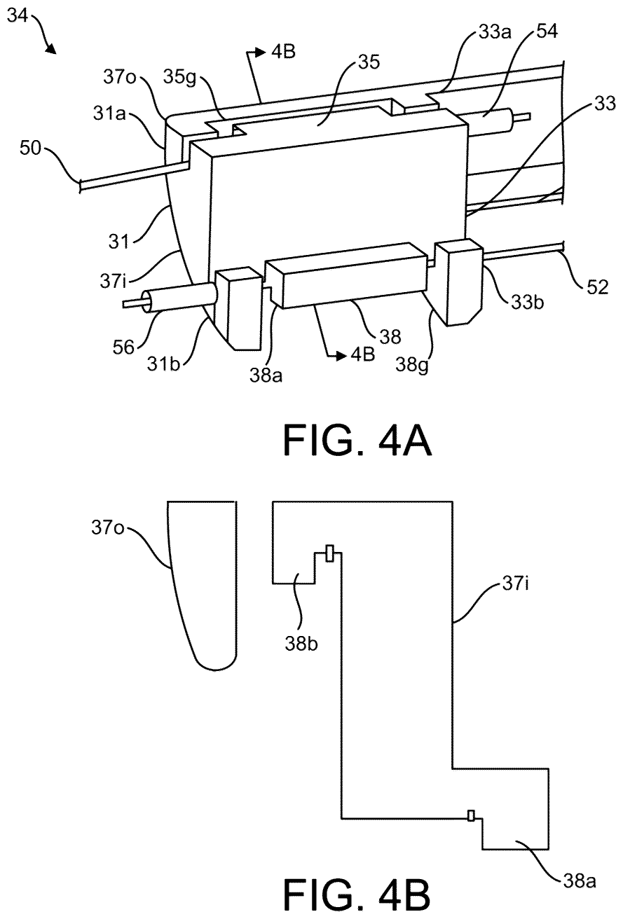

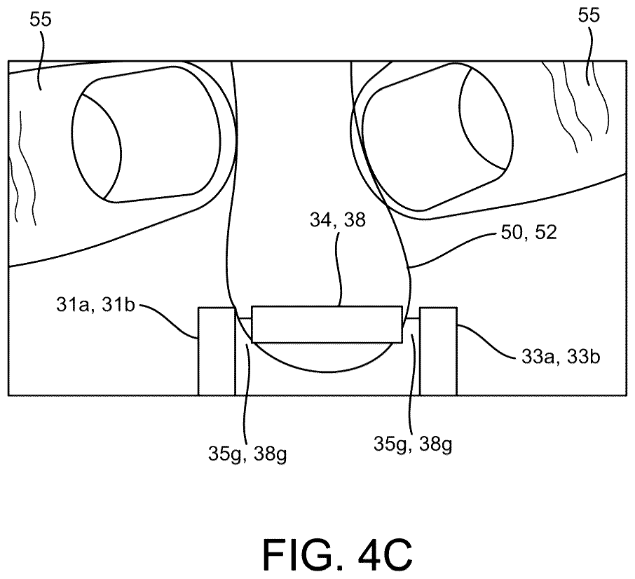

The wire retainer 34 is configured to receive and secure the distal ends of first and second control wires 50, 52. As shown in FIG. 4, the wire retainer 34 includes a distal face 31 and a proximal face 33. The wire retainer 34 includes a flat inner wall 37i and a curved outer wall 370 which extend between the distal face 31 and the proximal face 33. The curved outer wall 370 has a curvature that is similar to a curvature of the housing 20 and faces the housing 20. The flat inner wall 37i faces the sheath 90 along the longitudinal axis of the handle 100.

The distal face 31 includes a first channel 31a for receiving a portion of first control wire 50 and a second channel 31b for receiving a portion of second control wire 52. The proximal face 33 includes a first channel 33a for receiving a portion of first control wire 50 and a second channel 33b for receiving a portion of second control wire 52. The wire retainer 34 includes a first block 35 and a second block 38. As seen in FIG. 4B, the first block 35 and the second block 38 include an overhang 35a and 38a respectively. The first block 35 and the second block 38 extend partially between the distal face 31 and the proximal face such that gaps 35g, 38g are located on each end of the first block 35 and the second block 38. The first block 35 and the second block 38 each have a distal end and a proximal end adjacent gaps 35g, 38g.

The first block 35 in combination with the distal face first channel 31a and the proximal face first channel 33a form a passage that is configured to quickly receive and secure the first control wire 50. The first control wire 50 passes under first block 35 and sits in the distal face first channel 31a and the proximal face first channel 33a. A first control wire crimp 54 rests against the proximal face 33 of the wire retainer 34 and prevents the first control wire 50 from being pulled from the wire retainer 34. As the slide assembly 30 translates proximally within the handle 100, the first control wire crimp 54 presses against the proximal face 33 and tension is applied to the first control wire 50.

The second block 38 in combination with the distal face second channel 31b and the proximal face second channel 33b form a passage that is configured to quickly receive and secure the second control wire 52. The second control wire 52 passes under second block 38 and sits in the distal face second channel 31b and the proximal face second channel 33b. A second control wire crimp 56 rests against the distal face 31 of the wire retainer 34 and prevents the second control wire 52 from being pulled from the wire retainer 34. As the slide assembly 30 translates distally within the handle 100, the second control wire crimp 56 presses against the distal face 31 and tension is applied to the second control wire 52.

FIG. 4C illustrates how the first and second control wires 50, 52 are quickly inserted into and secured within the wire retainer 34. To insert the first and second controls wires 50, 52 into the wire retainer 34, the wire 50, 52 is grasped with fingers 55 of a user and flexed as illustrated. The flexed wire is inserted into the wire retainer 34 through gaps 35g, 39g and around the first block 35 or the second block 38 with the wire being held by the overhang 35a, 39a. The wire is then released and allowed to drop into the distal face first or second channel 31a, 32a and the proximal face first or second channel 31b, 33b. The wire 50, 52 is capable of sliding through the wire retainer 34 but the first control wire crimp 54 and the second control wire crimp 56 cannot pass the distal face 31 or the proximal face 33 respectively.

The handle 100 also includes EAM tubing 80 which is configured to receive one or more conductors that connect one or more electrodes associated with the sheath 90 to a control system. In some aspects, the control system is configured to deliver energy to the one or more electrodes to ablate tissue. In other aspects, the control system is configured to sense one or more parameters using the one or more electrodes.

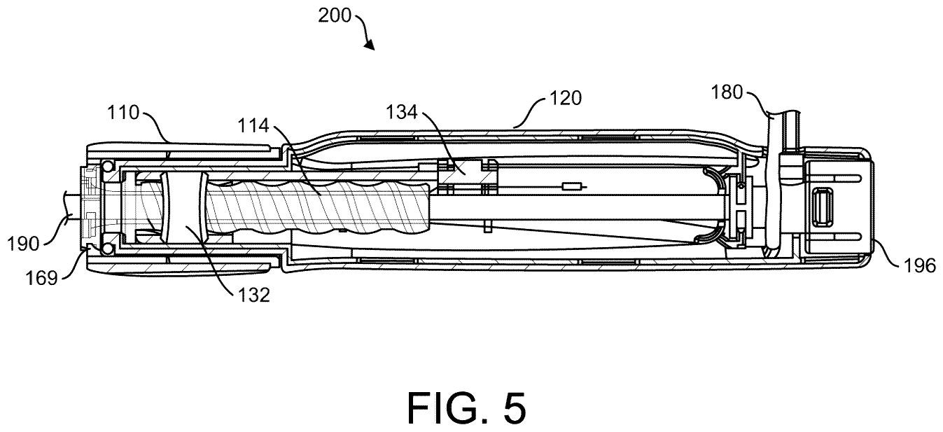

In one embodiment of the present disclosure as shown in FIG. 5, a steerable control system or handle 200 is provided for manipulating a medical device. The medical device may include, without limitation, a catheter, sheath, dilator, introducer, or similar medical devices. In a specific example, as shown, the handle 200 is coupled to a sheath 190 to enable a user to manipulate or steer the sheath 190 in a desired direction during use. The sheath 190 passes through a handle housing 120 from a proximal assembly 192 and extends distally from a knob 110 at distal end 121 of a handle housing 120.

The proximal assembly 192 includes a hub 196 having an aperture 198 that opens to a lumen that extends through the sheath 190. In one aspect, the aperture 198 is configured to receive an elongated medical device that can be inserted through the sheath 190, for example a guidewire, dilator, stylet, or other elongated medical device. In another aspect, the aperture 198 is configured to receive fluids, dyes, or medicaments that can be delivered to a desired location within a patient through the sheath 190.

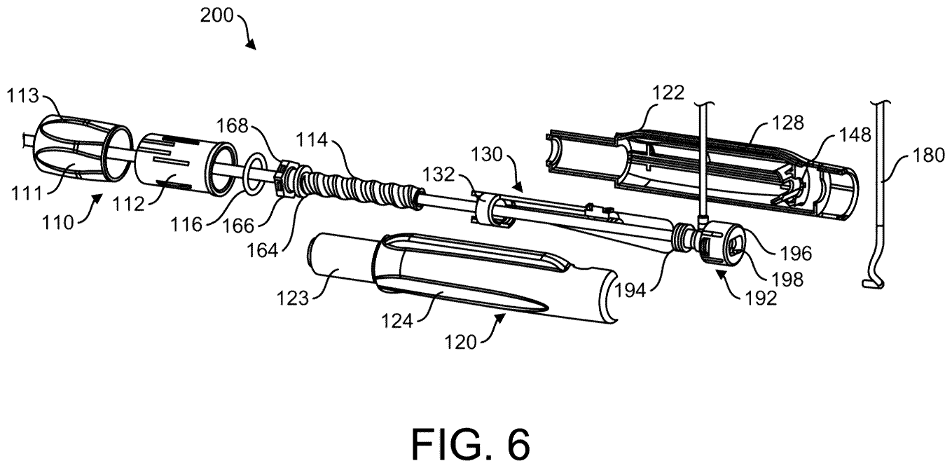

The proximal assembly 192 includes an annular recess 194. The annular recess 194 is configured to couple with corresponding shoulder 148 extending from an interior of handle housing 120. As shown in FIG. 6, handle housing 120 includes a right handle housing 122 and a left handle housing 124 which are configured to join together to encase components within the handle housing 120. The right handle housing 122 and the left handle housing 124 also include connecting elements 128, such as snap fit features, to allow the right handle housing 122 and the left handle housing 124 to be joined together.

The handle 200 comprises a knob 110 that is rotatably coupled to a distal portion 123 of the handle housing 120. The distal portion 123 includes a cylindrical extension, onto which the knob 110 is mounted. The knob 110 is rotatable about the longitudinal axis of the handle 200 and rotates with respect to housing 120. The knob 110 includes an external knob 111 and an internal knob 112. An external knob 111 can be connected to internal knob 112 and can include a plurality of raised portions 113 and be formed of a soft material to aid a user in gripping. The external knob 111 can remain fixed in place with adhesive, by a friction fit, or by mechanically coupling the external knob 111 to the internal knob 112, for example by one or more protrusions that couple with one or more slots or apertures. In operation, the rotation of the knob 110 in a first rotational direction allows the user to steer or deflect the sheath 190 in a first direction, whereas the rotation of the knob 110 in a second rotational direction allows the user to steer or deflect the sheath 190 in a second direction.

The rotation of the knob 110 is converted into a deflection of the sheath 190 via a slide assembly 130 and screw 114, shown in FIG. 6. Generally, knob 110 connects to a screw 114 that rotates as knob 110 is rotated. The screw 114 includes a distal portion including a flange 164 and a head 166. The head 166 is configured as a hexagon and is configured to connect with the internal knob 112. The head 166 includes a plurality of recesses 168 which can receive a plurality of extensions or protrusions 169 from the internal knob 112 to connect screw 114 to the internal knob 112. In one embodiment, the plurality of recesses 168 can be configured to snap fit with the plurality of extensions or protrusions 169. An O-ring 116 is located adjacent the head 166. The O-ring 116 is configured to reduce vibrations during rotation of the knob 110. The screw 114 include a lumen 119 that passes through the longitudinal axis thereof and sheath 90 passes through the lumen 119.

The screw 114 is co-operatively engaged with the slide assembly 130 which is housed within a lumen defined by the handle housing 120. The screw 114 includes a self-locking thread that communicates with a nut 132 of the slide assembly 130. As the knob 110 is rotated, the threads of the screw 114 cause translation of the slide assembly 130 via interaction with internal threads of the nut 132. In one embodiment, the slide assembly 130 translates proximally as the knob 110 is rotated in clockwise direction and distally when the knob 110 is rotated in a counter-clockwise direction. In another embodiment, the slide assembly 310 translates distally as the knob 110 is rotated in clockwise direction and proximally when the knob 110 is rotated in a counter-clockwise direction.

The slide assembly 130 includes the nut 132 at the distal end, a wire retainer 134 at the proximal end, and a bridge 136 joining the nut 132 and the wire retainer 134. The bridge 136 is configured as a partial cylinder and has a curved shape that partially circumscribes the screw 114 and sheath 190. Rotation of knob 110 causes a corresponding linear translation of the slide assembly 130 within the housing 120. This translation of the slide assembly 130 is converted into a tensioning of the control wires coupled to the slide assembly 130 and thereby resulting in a deflection of the sheath 190.

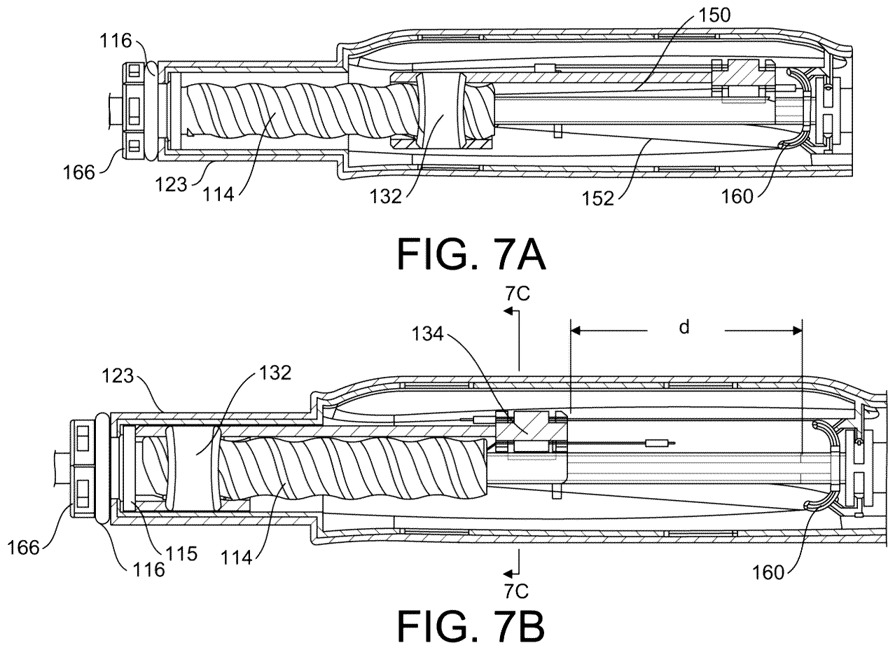

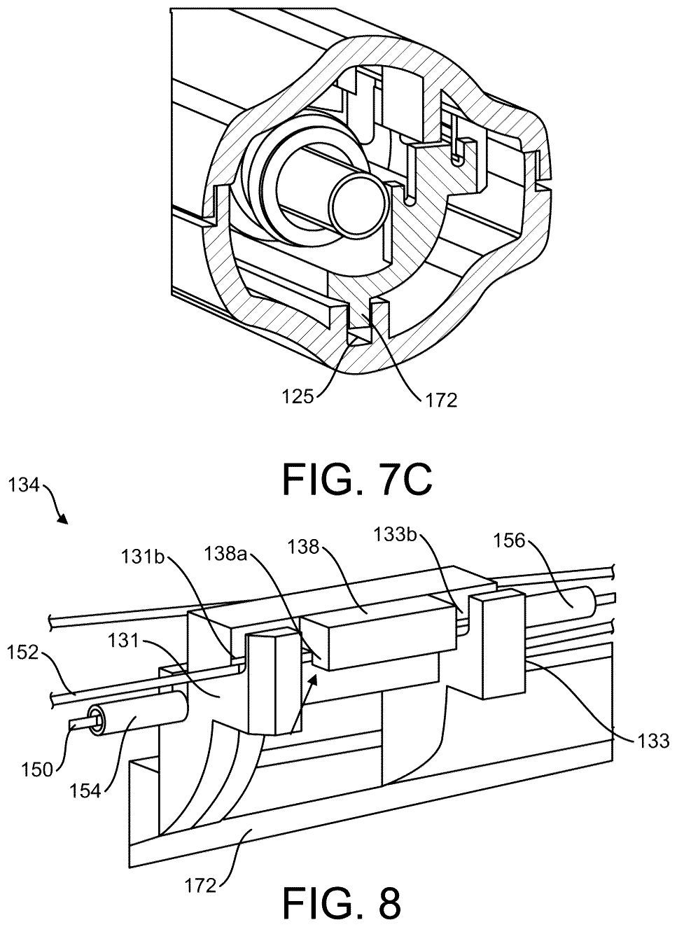

More specifically, slide assembly 130 is coupled to respective proximal ends of a pair of control wires that extend substantially along the length of the sheath 190, for example a first control wire 150 and a second control wire 152 as shown in FIG. 7A. A distal end (not shown) of each of the control wires 150, 152 is coupled to a distal portion of the sheath 190. The rotation of the knob 110 in one direction causes the slide assembly 130 to translate proximally within the housing 120 pulling one of the control wires (such as first control wire 150) to deflect the sheath 190 in a first direction, whereas the rotation of the knob 110 in an opposing direction causes the slide assembly 130 to translate distally within the housing 120 pulling the other of the control wires (such as second control wire 152) to deflect the sheath 190 in a second direction.

The amount of curvature imparted on the sheath 190 is limited by how far the slide assembly 130 translates within the handle 200. FIG. 7A is a partial view of handle 200 without the knob 110 and shows the slide assembly 130 in a most proximal position. The slide assembly 130 is limited distally by interaction of the nut 132 with flange 115 at a distal portion of screw 114. FIG. 7B is a partial view of handle 200 without the knob 110 and shows the slide assembly 130 in a most distal position. The slide assembly 130 is limited proximally by interaction of the wire retainer 134 with a projection within handle housing 120. In one embodiment, the slide assembly 130 is configured to translate within the housing 120 a distance d in the range of 1 to 3 inches. In one embodiment, the slide assembly 130 is configured to translate a distance d in the range of 1 to 1.5 inches. In another embodiment, the slide assembly 130 is configured to translate a distance d 1.38 inches.

FIG. 7C is a cross-sectional view through the wire retainer 134 of slide assembly 130 along line 7C in FIG. 7A. The right handle housing 122 includes a channel 125 that extends along the inner surface of right handle housing 122. As shown in FIG. 7C, the slide assembly 130 includes an extension 172 that fits within the channel 125 and remains within the channel 125 as the slide assembly 130 translates along the handle 200. This prevents rotation of the slide assembly 130 during rotation of the screw 114.

In one example as shown in FIGS. 7A and 7B, in order to allow the slide assembly 130 to separately impart a pulling force on each of the two control wires, one of the two control wires (such as first control wire 150) is directly coupled to the wire retainer 134 of slide assembly 130 whereas the other of the control wires (such as second control wire 152) is indirectly coupled to the slide assembly 130 via a direction reversing element 160 such a pulley or pin. In some embodiments, the reversing element 160 may include a curved guide, channel, or wall through which the second control wire 152 passes. In other words, a means for of coupling the distal ends of the wires 150, 152 to opposite sides of the sheath 190 is included in the handle, whereby motion of the slide assembly 130 in one direction will apply tension to one wire while motion of the slide in the other direction will apply tension to the other wire. As used herein, “directly coupled” is taken to mean that the proximal end of the wire is operably coupled (but not necessarily physically attached or integral with) to the slide assembly 130 without passing through an intermediate structure, while “indirectly coupled” is taken to mean that the proximal end of the wire is operably coupled (but not necessarily physically attached or integral with) to the slide assembly 130 after passing through an intermediate structure or element, such as a direction reversing element 160.

In one aspect, a proximal end of first control wire 150 exits sheath 190 and is coupled at or to the slide assembly 130 at the wire retainer 134. Thus, in this example, first control wire 150 is directly coupled to slide assembly 130 via the wire retainer 134. Similarly, a proximal end of second control wire 152 exits sheath 190 and is routed proximally. The second control wire 152 is then passed around or through the direction reversing element 160 and routed back distally so that it can be passed distally through the slide assembly 130 to be coupled at or to a distal face 131 of the wire retainer 134, i.e., distally of the slide assembly. Thus, in this example, second control wire 152 is indirectly coupled to the slide assembly 130.

The wire retainer 134 is configured to receive and secure the distal ends of first and second control wires 150, 152. As shown in FIG. 8, the wire retainer 134 includes a distal face 131 and a proximal face 133. The distal face 131 includes a first channel 131a for receiving a portion of first control wire 150 and a second channel 131b for receiving a portion of second control wire 152. The proximal face 133 includes a first channel 133a for receiving a portion of first control wire 150 and a second channel 133b for receiving a portion of second control wire 152. The wire retainer 134 includes a first block 135 and a second block 138. The first block 135 and the second block 138 include an overhang 135a and 138a respectively. The first block 135 and the second block 138 extend partially between the distal face 131 and the proximal face such that gaps 135g, 138g are located on each end of the first block 135 and the second block 138.

The first block 135 in combination with the distal face first channel 131a and the proximal face first channel 133a form a passage that is configured to quickly receive and secure the first control wire 150. The first control wire 150 passes under first block 135 and sits in the distal face first channel 131a and the proximal face first channel 133a. A first control wire crimp 154 rests against the proximal face 133 of the wire retainer 134 and prevents the first control wire 150 from being pulled from the wire retainer 134. As the slide assembly 130 translates proximally within the handle 200, the first control wire crimp 154 presses against the proximal face 133 and tension is applied to the first control wire 150.

The second block 138 in combination with the distal face second channel 131b and the proximal face second channel 133b form a passage that is configured to quickly receive and secure the second control wire 152. The second control wire 152 passes under second block 138 and sits in the distal face second channel 131b and the proximal face second channel 133b. A second control wire crimp 156 rests against the distal face 131 of the wire retainer 134 and prevents the second control wire 152 from being pulled from the wire retainer 134. As the slide assembly 130 translates distally within the handle 200, the second control wire crimp 156 presses against the distal face 131 and tension is applied to the second control wire 152. The first and second control wires 150, 152 can be quickly inserted into and secured within the wire retainer 134 in a manner as described in FIG. 4C above.

The handle 200 also includes EAM tubing 180 which is configured to receive one or more conductors that connect one or more electrodes associated with the sheath 90 to a control system. In some aspects, the control system is configured to deliver energy to the one or more electrodes to ablate tissue. In other aspects, the control system is configured to sense on or more parameters using the one or more electrodes.

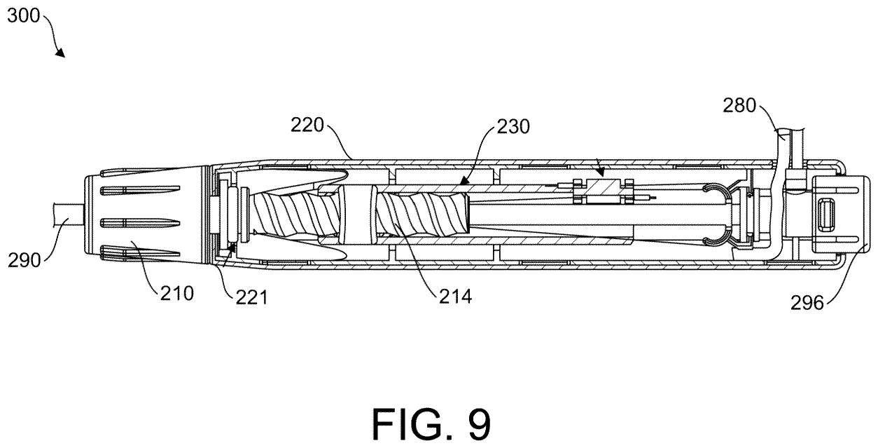

In one embodiment of the present disclosure as shown in FIG. 9, a steerable control system or handle 300 is provided for manipulating a medical device. The medical device may include, without limitation, a catheter, sheath, dilator, introducer, or similar medical devices. In a specific example, as shown, the handle 300 is coupled to a sheath 290 to enable a user to manipulate or steer the sheath 90 in a desired direction during use. The sheath 290 passes through a handle housing 220 from a proximal assembly 292 and extends distally from a distal end 221 of a handle housing 220.

The proximal assembly 292 includes a hub 296 having an aperture 298 that opens to a lumen that extends through the sheath 290. In one aspect, the aperture 298 is configured to receive an elongated medical device that can be inserted through the sheath 290, for example a guidewire, dilator, stylet, or other elongated medical device. In another aspect, the aperture 298 is configured to receive fluids, dyes, or medicaments that can be delivered to a desired location within a patient through the sheath 290.

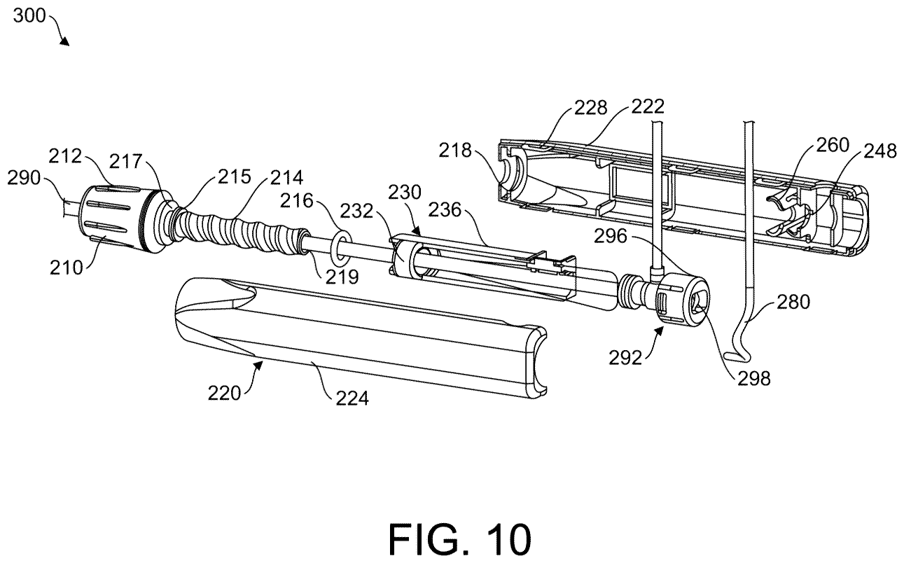

The proximal assembly 292 includes an annular recess 294. The annular recess 294 is configured to be held by an annular protrusion 248 extending from an inner surface of handle housing 220. As shown in FIG. 10, the handle housing 220 includes a right handle housing 222 and a left handle housing 224 which are configured to join together to encase components within the handle housing 220. The right handle housing 222 and the left handle housing 224 also include connecting elements 228, such as snap fit features, to allow the right handle housing 222 and the left handle housing 224 to be joined together.

The handle 300 comprises a knob 210 that is rotatably coupled to a distal end 221 of the handle housing 220. The knob 210 is rotatable about the longitudinal axis of the handle 300 and rotates with respect to housing 220. In operation, the rotation of the knob 210 in a first rotational direction allows the user to steer or deflect the sheath 290 in a first direction, whereas the rotation of the knob 210 in a second rotational direction allows the user to steer or deflect the sheath 290 in a second direction.

The rotation of the knob 210 is converted into a deflection of the sheath 290 via a slide assembly 230 and screw 214. Generally, knob 210 includes an integrally molded screw 214 that rotates as knob 210 is rotated. In one embodiment, knob 210 can be formed of a softer material that is overmolded onto a portion of the screw 214 that extends out of housing 220. The knob 120 may include a plurality of raised portions 212 that aid a user in gripping the knob 210. An O-ring 216 sits in a channel 217 on the screw 214 and interacts with an annular projection 218 extending from an internal surface of the housing 220. The O-ring 216 is configured to dampen vibrations and provide a smooth feel as the knob 210 is rotated. The knob 210 and screw 214 include a lumen 219 that passes through the longitudinal axis thereof.

The screw 214 is co-operatively engaged with the slide assembly 230 which is housed within a lumen defined by the handle housing 220. The screw 214 includes a self-locking thread that communicates with a nut 232 of the slide assembly 230. As the knob 210 is rotated, the threads of the screw 214 cause translation of the slide assembly 230 via interaction with internal threads of the nut 232. In one embodiment, the slide assembly 230 translates proximally as the knob 210 is rotated in clockwise direction and distally when the knob 210 is rotated in a counter-clockwise direction. In another embodiment, the slide assembly 230 translates distally as the knob 210 is rotated in clockwise direction and proximally when the knob 210 is rotated in a counter-clockwise direction.

The slide assembly 230 includes the nut 232 at the proximal end, a wire retainer 234 at the distal end, and a bridge 236 joining the nut 232 and the wire retainer 234. The bridge 236 includes a curved inner surface and partially surrounds the screw 214 and sheath 290. The rotation of knob 210 causes a corresponding linear translation of the slide assembly 230 within the housing 220. This translation of the slide assembly 230 is converted into a tensioning of the control wires coupled to the slide assembly 230 and thereby resulting in a deflection of the sheath 290.

More specifically, slide assembly 230 is coupled to respective proximal ends of a pair of control wires that extend substantially along the length of the sheath 290, for example a first control wire 250 and a second control wire 252 as shown in FIG. 10. A distal end (not shown) of each of the control wires 250, 252 is coupled to a distal portion of the sheath 290. The rotation of the knob 210 in one direction causes the slide assembly 230 to translate proximally within the housing 220 pulling one of the control wires (such as first control wire 250) to deflect the sheath 290 in a first direction, whereas the rotation of the knob 210 in an opposing direction causes the slide assembly 230 to translate distally within the housing 220 pulling the other of the control wires (such as second control wire 252) to deflect the sheath 290 in a second direction.

The amount of curvature imparted on the sheath 290 is limited by how far the slide assembly 230 translates within the handle 300. The slide assembly 230 is limited proximally by interaction of the nut 232 with flange 215 forming a portion of channel 217 to hold O-ring 216. The slide assembly 30 is limited distally by interaction of the wire retainer 234 with a direction reversing element 260. In one embodiment, the slide assembly 230 is configured to translate a distance d in the range of 1 to 4 inches. In one embodiment, the slide assembly 230 is configured to translate a distance d in the range of 1 to 2 inches.

As shown in FIG. 10, to allow the slide assembly 230 to separately impart a pulling force on each of the two control wires, one of the two control wires (such as first control wire 250) is directly coupled to the wire retainer 234 of slide assembly 230 whereas the other of the control wires (such as second control wire 252) is indirectly coupled to the slide assembly 230 via a direction reversing element 260 such a pulley or a pin. In some embodiments, the reversing element 260 may include a curved guide, channel, or wall through which the second control wire 252 passes. In other words, a means for of coupling the distal ends of the wires 250, 252 to opposite sides of the sheath 290 is included in the handle, whereby motion of the slide assembly 230 in one direction will apply tension to one wire while motion of the slide in the other direction will apply tension to the other wire. As used herein, directly coupled is taken to mean that the proximal end of the wire is operably coupled (but not necessarily physically attached or integral with) to the slide assembly 230 without passing through an intermediate structure, while indirectly coupled is taken to mean that the proximal end of the wire is operably coupled (but not necessarily physically attached or integral with) to the slide assembly 230 after passing through an intermediate structure or element, such as a direction reversing element 260.

In one aspect, a proximal end of first control wire 250 exits sheath 290 and is coupled at or to the slide assembly 230 at the wire retainer 234. Thus, in this example, first control wire 250 is directly coupled to slide assembly 230 via the wire retainer 234. Similarly, a proximal end of second control wire 252 exits sheath 290 and is routed proximally. The second control wire 252 is then passed around or through the direction reversing element 260 and routed back distally so that it can be passed distally through the slide assembly 230 to be coupled at or to a distal face 231 of the wire retainer 234, i.e., distally of the slide assembly. Thus, in this example, second control wire 252 is indirectly coupled to the slide assembly 230.

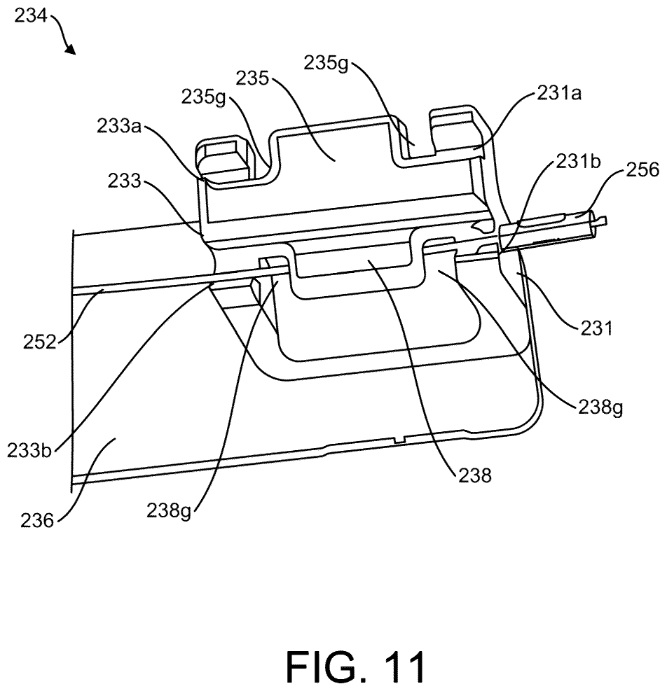

The wire retainer 234 is configured to receive and secure the distal ends of first and second control wires 250, 252. As shown in FIG. 11, the wire retainer 234 includes a distal face 231 and a proximal face 233. The distal face 231 includes a first channel 231a for receiving a portion of first control wire 250 and a second channel 231b for receiving a portion of second control wire 252. The proximal face 233 includes a first channel 233a for receiving a portion of first control wire 250 and a second channel 233b for receiving a portion of second control wire 252. The wire retainer 234 includes a first block 235 and a second block 238. The first block 235 and the second block 238 include an overhang 235a and 238a respectively. The first block 235 and the second block 238 extend partially between the distal face 231 and the proximal face 233 such that gaps 235g, 238g are located on each end of the first block 235 and the second block 238.

The first block 235 in combination with the distal face first channel 231a and the proximal face first channel 233a form a passage that is configured to quickly receive and secure the first control wire 250. The first control wire 250 passes under first block 235 and sits in the distal face first channel 231a and the proximal face first channel 233a. A first control wire crimp 254 rests against the proximal face 233 of the wire retainer 234 and prevents the first control wire 250 from being pulled from the wire retainer 234. As the slide assembly 230 translates proximally within the handle 300, the first control wire crimp 254 presses against the proximal face 233 and tension is applied to the first control wire 250.

The second block 238 in combination with the distal face second channel 231b and the proximal face second channel 233b form a passage that is configured to quickly receive and secure the second control wire 252. The second control wire 252 passes under second block 238 and sits in the distal face second channel 231b and the proximal face second channel 233b. A second control wire crimp 256 rests against the distal face 231 of the wire retainer 234 and prevents the second control wire 252 from being pulled from the wire retainer 234. As the slide assembly 230 translates distally within the handle 300, the second control wire crimp 256 presses against the distal face 31 and tension is applied to the second control wire 252. The first and second control wires 250, 252 can be quickly inserted into and secured within the wire retainer 234 in a manner as described in FIG. 4C above.

The handle 300 also includes EAM tubing 280 which is configured to receive one or more conductors that connect one or more electrodes associated with the sheath 290 to a control system. In some aspects, the control system is configured to deliver energy to the one or more electrodes to ablate tissue. In other aspects, the control system is configured to sense on or more parameters using the one or more electrodes.

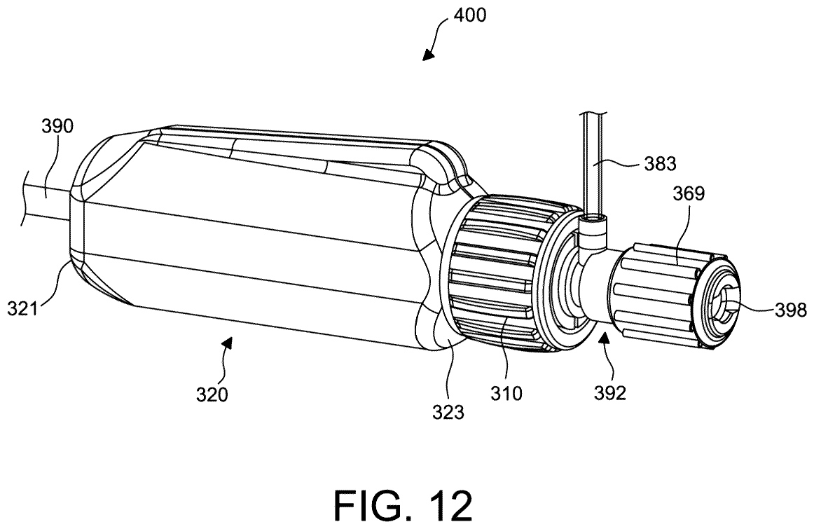

In another embodiment of the present disclosure as shown in FIG. 12, a steerable control system or handle 400 is provided for manipulating a medical device. The medical device may include, without limitation, a catheter, sheath, dilator, introducer, or similar medical devices. In a specific example, as shown, the handle 400 is coupled to a sheath 390 to enable a user to manipulate or steer the sheath 390 in a desired direction during use. The sheath 390 passes through a handle housing 320 from a proximal assembly 392 and extends distally from a distal end 321 of a handle housing 320.

An aperture 398 located at the proximal end of the proximal assembly 392 opens to a lumen that extends through the sheath 390. In one aspect, the aperture 398 is configured to receive an elongated medical device that can be inserted through the sheath 390, for example a guidewire, dilator, stylet, or other elongated medical device. In another aspect, the aperture 398 is configured to receive fluids, dyes, or medicaments that can be delivered to a desired location within a patient through the sheath 390.

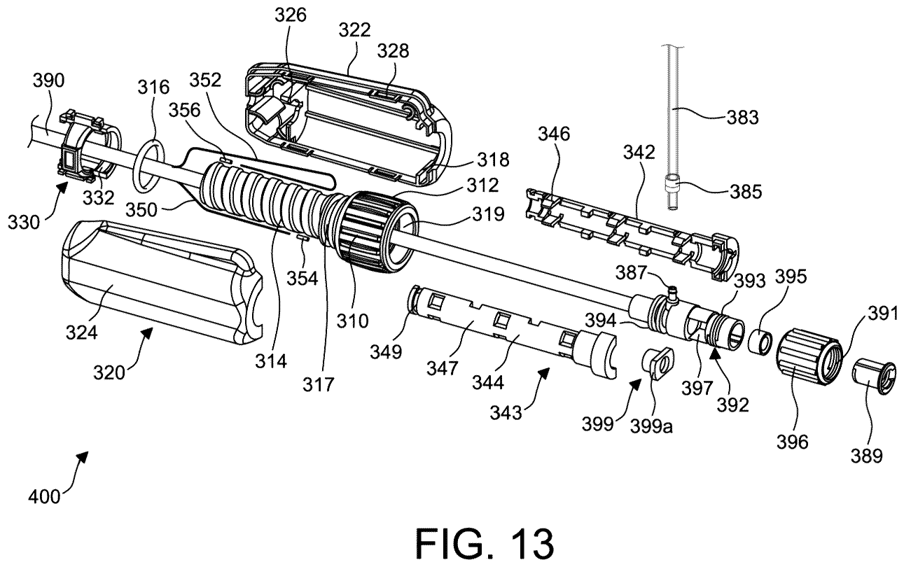

As illustrated in FIG. 13, the proximal assembly 392 includes a nut 396 having internal threads 391. The nut 396 is configured to translate over a proximal portion of the proximal assembly 392. Internal threads 391 mate with external threads 393 on the proximal assembly. Rotation of the nut 396 allows for opening and closing of a valve 395 located within a proximal channel 397 of the proximal assembly 392. The valve 395 allows for introduction of a medical device or fluid into the sheath 390 from the aperture 398.

The valve 395 is opened and closed by rotation of the nut 396, which translates a compressor 399 that is located proximally of the valve 395 in the proximal channel 397. The compressor 399 includes a flange 399a that is held within recess or engaged with a shoulder within the interior of the nut 396. As such, as the nut 395 is rotated in a first direction, the compressor 399 is translated distally and compresses the valve 395. This causes the valve 395 to close. As the nut 396 is rotated in a second direction, the compressor 399 is translated proximally and allows the valve 395 to decompress. Thus, allowing the valve 395 to open. In another embodiment, the compressor 399 is not held by the nut 396. Rather, the compressor 399 freely moves within the nut 396. When the nut 396 is rotated in a first direction, the compressor 399 is pushed against the valve 395 compressing the valve 395. When the nut 396 is rotated in a second direction, space is created between the compressor 399 and the nut 396 providing the compressor 399 room to move. This allows the valve 395 to expand, thus opening, and push the compressor 399 proximally. A snap 389 is positioned proximally of the compressor 399 in the proximal channel 397 of the proximal assembly 392. The snap 389 locks into the proximal channel 397 and defines the aperture 398.

As illustrated in FIG. 13, the proximal assembly 392 includes an annular shoulder 394. The annular shoulder 394 is configured to be held by a recess 348 formed by a right hub holder 342 and a left hub holder 344 when assembled. The right hub holder 342 and the left hub holder 344 are configured to connect together using connecting elements 346, such as snap fit features to form a hub holder 343.

The proximal assembly 392 also includes a port 387. Port 387 connects with flexible tubing 383 and allows an additional channel for the introduction of fluids into or out of the sheath 390. A tube clamp 385 ensures the flexible tubing 383 remains secured to port 387 when connected thereto.

As shown in FIG. 13, the handle housing 320 includes a right handle housing 322 and a left handle housing 324 which are configured to join together to encase components within the handle housing 320. The right handle housing 322 and the left handle housing 324 include a hub holder support 326 configured to retain a distal end of the hub holder 343. The hub holder support 326 fits within an annular recess 349 at the distal end of hub holder 343. The hub holder support 326 ensures the longitudinal axis of a screw 314 that is supported by hub holder 343 remains centered along the longitudinal axis of the handle 400. The right handle housing 322 and the left handle housing 324 also include connecting elements 328, such as snap fit features, to allow the right handle housing 322 and the left handle housing 324 to be joined together.

The handle 400 comprises a knob 310 that is rotatably coupled to a proximal end 323 of the handle housing 320. The knob 310 is rotatable about the longitudinal axis of the handle 400 and rotates with respect to handle housing 320. In operation, the rotation of the knob 310 in a first rotational direction allows the user to steer or deflect the sheath 390 in a first direction, whereas the rotation of the knob 310 in a second rotational direction allows the user to steer or deflect the sheath 390 in a second direction.

The rotation of the knob 310 is converted into a deflection of the sheath 390 via a slide assembly 330 and screw 314. Generally, knob 310 includes an integrally molded screw 314 that rotates as knob 310 is rotated. In one embodiment, knob 310 can be formed of a softer material that is overmolded onto a portion of the screw 314 that extends out of housing 320. The knob 310 may include a plurality of raised portions 312 that aid a user in gripping the knob 310. An O-ring 316 sits in a channel 317 on the screw 314 and interacts with a projection 318 extending from an internal surface of the housing 320. The O-ring 316 is configured to dampen vibrations and provide a smooth feel as the knob 310 is rotated.

The knob 310 and screw 314 include a lumen 319 that passes through the longitudinal axis thereof. The hub holder 343 includes an elongate cylindrical body 347 that is configured to pass through the lumen 319 and support the knob 310 and screw 314. Sheath 390 passes through an interior lumen of the hub holder 343 and into the proximal assembly 392.

The screw 314 is co-operatively engaged with the slide assembly 330 which is housed within the handle housing 320. The screw 314 includes a self-locking thread that communicates with internal threads 332 of the slide assembly 330. As the knob 310 is rotated, the threads of the screw 314 cause translation of the slide assembly 330 via interaction with internal threads 332. In one embodiment, the slide assembly 330 translates proximally as the knob 310 is rotated in clockwise direction and distally when the knob 310 is rotated in a counter-clockwise direction. In another embodiment, the slide assembly 330 translates distally as the knob 310 is rotated in clockwise direction and proximally when the knob 310 is rotated in a counter-clockwise direction.

The threads of the screw 314 can include a thread pitch in the range of 0.25″ to 0.45″. In one embodiment, the thread pitch is 0.333″. The threads may include an angle in relation to the surface of the screw 314 in the range of 90 degrees to 110 degrees. In one embodiment, the angle is 100 degrees. The pitch diameter of the threads of the screw 314 can be in the range of 0.5″ to 0.7″. In one embodiment, the pitch diameter is 0.647″. The threads of the screw 314 can be configured such that between 3 complete turns of the knob 310 and 5 complete turns of the knob 310 translates the slide assembly 330 from a most proximal position to a most distal position. In one embodiment, the threads are configured such that 4.1 turns of the knob 310 can move the slide assembly 330 from a most proximal position to the most distal position or vice versa. The threads of the screw 314 are designed to be self-locking. A coefficient of friction greater than 0.11 ensures that the threads are self-locking.

In use, rotation of knob 310 causes a corresponding linear translation of the slide assembly 330 within the housing 320. The slide assembly 330 is coupled to at least one control wire associated with a distal portion of sheath 390. The translation of the slide assembly 330 is converted into a tensioning of the at least one control wire coupled to the slide assembly 330 and thereby resulting in a deflection of the sheath 390.

More specifically, slide assembly 330 is coupled to respective proximal ends of a pair of control wires that extend substantially along the length of the sheath 390, for example a first control wire 350 and a second control wire 352. A distal end (not shown) of each of the control wires 350, 352 is coupled to a distal portion of the sheath 390. The rotation of the knob 310 in one direction causes the slide assembly 330 to translate proximally within the housing 320 pulling one of the control wires (such as first control wire 350) to deflect the sheath 390 in a first direction, whereas the rotation of the knob 310 in an opposing direction causes the slide assembly 330 to translate distally within the housing 320 pulling the other of the control wires (such as second control wire 352) to deflect the sheath 390 in a second direction. While slide assembly 330 applies tension to the first control wire 350, the second control wire 352 is free to move unencumbered through the slide assembly 330. Similarly, as the slide assembly 330 applies tension to the second control wire 352, the first control wire 350 is free to move unencumbered through the slide assembly 330. As such, tension is only applied to one of the first control wire 350 or the second control wire 352 at any time.

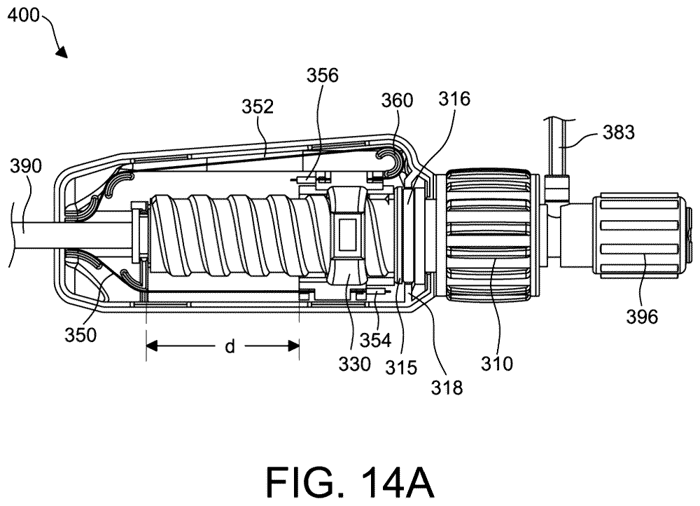

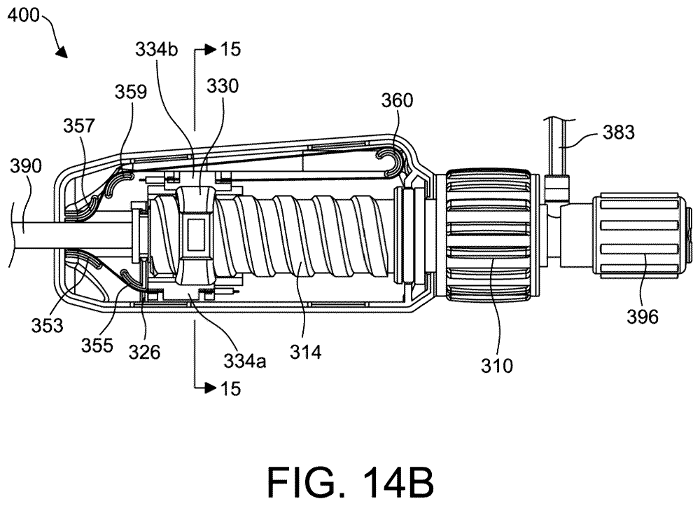

The amount of curvature imparted on the sheath 390 is limited by how far the slide assembly 330 translates within the handle 400. FIG. 14A shows the slide assembly 330 in a most proximal position. The slide assembly 330 is limited proximally by interaction with flange 315 forming a portion of channel 317 to hold O-ring 316. FIG. 14B shows the slide assembly 330 in a most distal position. The slide assembly 330 is limited distally by interaction with hub holder support 326. In one embodiment, the slide assembly 330 is configured to translate within the handle 400 a distance d in the range of 1 to 2 inches. In one embodiment, the slide assembly 330 is configured to translate a distance d in the range of 1 to 1.5 inches. In another embodiment, the slide assembly 330 is configured to translate a distance d of 1.37 inches.

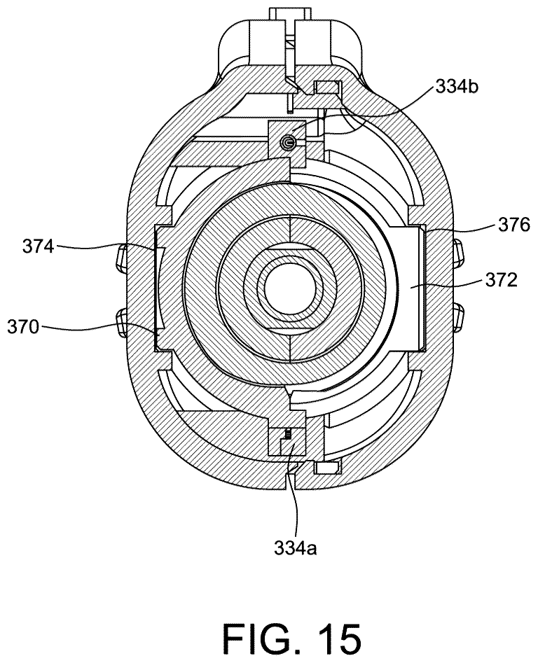

FIG. 15 is a cross-sectional view through the slide assembly 330 along line 15 in FIG. 14B. As shown, the slide assembly 330 includes a first raised portion 370 and a second raised portion 372 that is configured to translate within a first guide 374 and a second guide 376 respectively. The first guide 374 and the second guide 376 extend along the inner portion of right handle housing 322 and the left handle housing 324. The first guide 374 and the second guide 376 have a shape that corresponds to the shape of the first raised portion 370 and the second raised portion 372. In one embodiment, the first raised portion 370 and the second raised portion 372 are substantially rectangular as shown FIGS. 14A and 41B. The interaction of the raised portions 370, 372 and the guides 374, 376 allows the slide assembly 330 to translate along the handle 400 without rotating while knob 310 is rotated.

The slide assembly 330 includes a first wire retainer 334a located at the bottom of the slide assembly 330 and a second wire retainer 334b located at a top of the slide assembly 330. The first wire retainer 334a and the second wire retainer 334b are positioned on opposite sides of the slide assembly 330. In one example as shown in FIGS. 14A and 14B, in order to allow the slide assembly 330 to separately impart a pulling force on each of the two control wires, one of the two control wires (such as first control wire 350) is coupled to the first wire retainer 334a of the slide assembly 330 whereas the other of the control wires (such as second control wire 352) is coupled to the second wire retainer 334b of the slide assembly 330.

The first control wire 350 exits the sheath 390 within the housing 320 and contacts a first control wire first guide 353 and a first control wire second guide 355 before coupling with the first wire retainer 334a. First control wire first guide 353 and first control wire second guide 355 can include a static or movable bearing, pulley, post, pin, curved wall, or channel to position the first control wire 350 in a desired orientation. First control wire first guide 353 and first control wire second guide 355 position the first wire 350 such that the first wire 350 remains substantially parallel to a longitudinal axis of the screw 314 proximal of the first control wire second guide 355. The first control wire 350 is coupled to the first wire retainer 334a such that proximal movement of the slide assembly 330 causes tension on the first control wire 350, allowing deflection of the sheath 390 in a first direction.

The second control wire 352 exists the sheath 390 within the housing 320 and contacts a second control wire first guide 357, a second control wire second guide 359, and a reversing element 360 before coupling with the second wire retainer 334b. The second control wire first guide 357, second control wire second guide 359, and the reversing element 360 may include a static or movable bearing, pulley, post, pin, curved guide, wall, or channel through or around which the second control wire 352 passes to position the second control wire 352 in a desired orientation. Second control wire first guide 357, second control wire second guide 359, and reversing element 360 position the second wire 352 such that the second wire 352 remains substantially parallel to a longitudinal axis of the screw 314 distal of the reversing element 360.

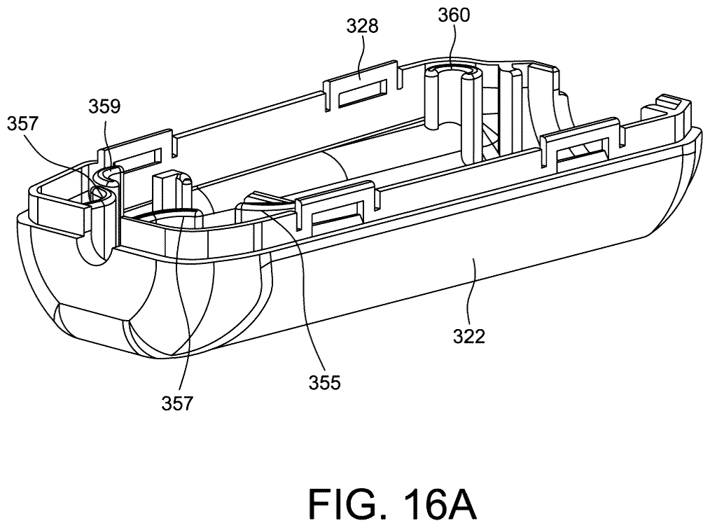

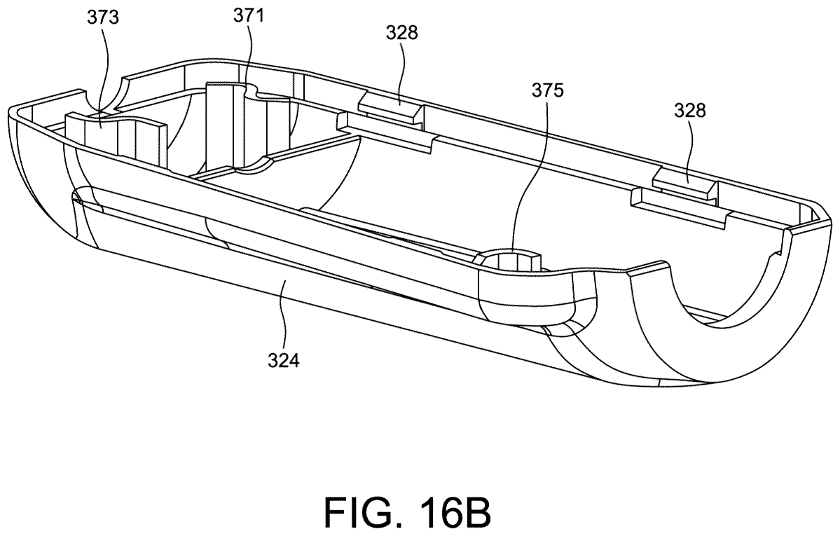

In one embodiment, the first control wire first guide 353, the first control wire second guide 355, the second control wire first guide 357, second control wire second guide 359, and the reversing element 360 each extend from an inner surface of the right handle housing 322 as seen in FIG. 16A.

As shown in FIG. 16B, the left handle housing 324 includes a first guide rib 371, a second guide rib 373, and a third guide rib 375 extending from an inner surface thereof. The first guide rib 371, second guide rib 373, and third guide rib 375 are configured such that upon assembly of the right handle housing 322 and the left handle housing 324, the first guide rib 371, second guide rib 373, and third guide rib 375 constrain the first control wire 350 and the second control wires 352 in a plane passing through the longitudinal axis of the screw 314.

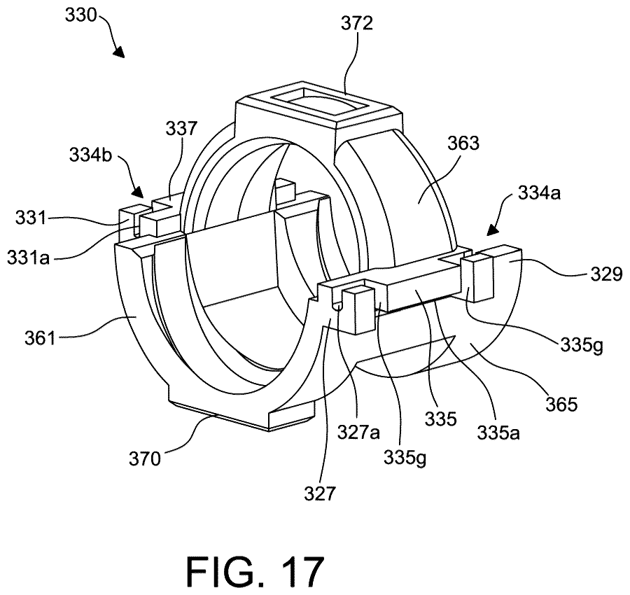

As shown in FIG. 17, the slide assembly 330 includes three sections forming a generally ring-shape. The first section 361, second section 363, and third section 365 are each approximately half of a circle arranged such that the ends connect to form a complete ring. The first section 361 is separated from the third section 365 by the second section 363. The first section 361 and the third section 365 are each located on a first half of the slide assembly 330. The second section 363 forms the second half of the slide assembly 330. The first section 361, second section 363, and third section 365 each include an inner surface and an outer surface. The inner surface of each section includes internal threads that interact with the threads of the screw 314. The outer surface of the first section 361 includes the first raised portion 370. The second raised portion 372 is located on the outer surface of the second section 363. In one embodiment, the third second 365 includes an additional raised portion.

The first wire retainer 344a and the second wire retainer 344b span across the outer surface of the first section 361, the second section 363, and the third section 365. The first wire retainer 344a and the second wire retainer 344b are configured to receive and secure the distal ends of first and second control wires 350, 352 respectively.

As shown in FIG. 17, the first wire retainer 344a includes a distal face 327 and a proximal face 329. The distal face 327 includes a channel 327a for receiving a portion of the first control wire 350. The proximal face 329 includes a channel 329a for receiving a portion of first control wire 350. The first wire retainer 344a includes a first wire retainer block 335. The first wire retainer block 335 includes an overhang 335a. The first wire retainer block 335 extends partially between the distal face 327 and the proximal face 329 such that gaps 335g are located on each end of the first wire retainer block 335. The first wire retainer block 335 has a distal end and a proximal end adjacent gaps 335g.

The first wire retainer block 335 in combination with the distal face channel 327a and the proximal face channel 329a form a passage that is configured to quickly receive and secure the first control wire 350. The first control wire 350 passes under first wire retainer block 335 and sits in the distal face channel 327a and the proximal face channel 329a. A first control wire crimp 354 rests against the proximal face 329 of the first wire retainer 334a and prevents the first control wire 350 from being pulled from the first wire retainer 334a. As the slide assembly 330 translates proximally within the handle 400, the first control wire crimp 354 presses against the proximal face 329 and tension is applied to the first control wire 350.

The second wire retainer 344b includes a distal face 331 and a proximal face 333. The distal face 331 includes a channel 331a for receiving a portion of the second control wire 352. The proximal face 333 includes a channel 333a for receiving a portion of the second control wire 352. The second wire retainer 344b includes a second wire retainer block 337. The second wire retainer block 337 includes an overhang 337a. The second wire retainer block 337 extends partially between the distal face 331 and the proximal face 333 such that gaps 337g are located on each end of the second wire retainer block 337. The second wire retainer block 337 has a distal end and a proximal end adjacent gaps 337g.

The second wire retainer block 337 in combination with the distal face channel 331a and the proximal face channel 333a form a passage that is configured to quickly receive and secure the second control wire 352. The second control wire 352 passes under second wire retainer block 337 and sits in the distal face channel 331a and the proximal face channel 333a. A second control wire crimp 356 rests against the distal face 331 of the second wire retainer 344b and prevents the second control wire 352 from being pulled from the second wire retainer 334b. As the slide assembly 330 translates distally within the handle 400, the second control wire crimp 356 presses against the distal face 331 and tension is applied to the second control wire 352. First and second control wires 350, 352 are quickly inserted into and secured within the wire retainer 334 as illustrated and described above in FIG. 4C.

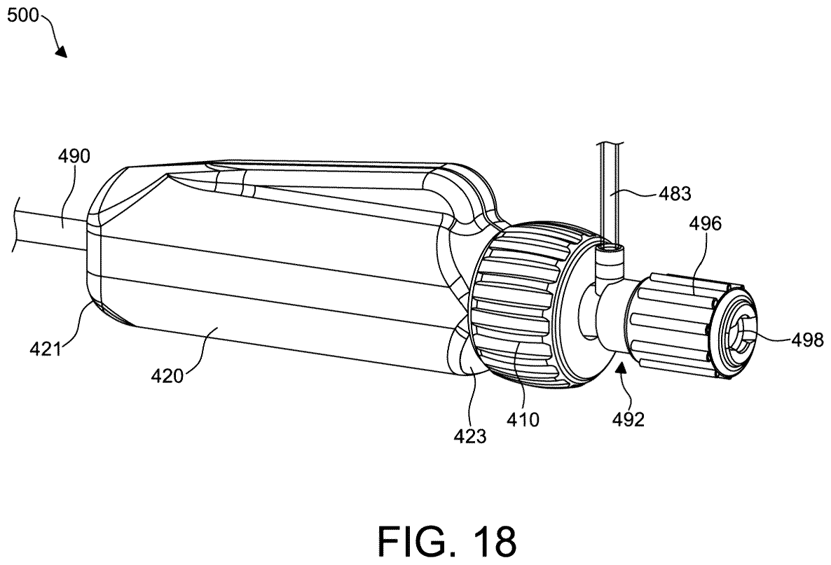

In another embodiment of the present disclosure as shown in FIG. 18, a steerable control system or handle 500 is provided for manipulating a medical device. The medical device may include, without limitation, a catheter, sheath, dilator, introducer, or similar medical devices. In a specific example, as shown, the handle 500 is coupled to a sheath 490 to enable a user to manipulate or steer the sheath 490 in a desired direction during use. The sheath 490 passes through a handle housing 420 from a proximal assembly 492 and extends distally from a distal end 421 of a handle housing 420.

An aperture 498 located at the proximal end of the proximal assembly 392 opens to a lumen that extends through the sheath 490. In one aspect, the aperture 498 is configured to receive an elongated medical device that can be inserted through the sheath 490, for example a guidewire, dilator, stylet, or other elongated medical device. In another aspect, the aperture 498 is configured to receive fluids, dyes, or medicaments that can be delivered to a desired location within a patient through the sheath 490.

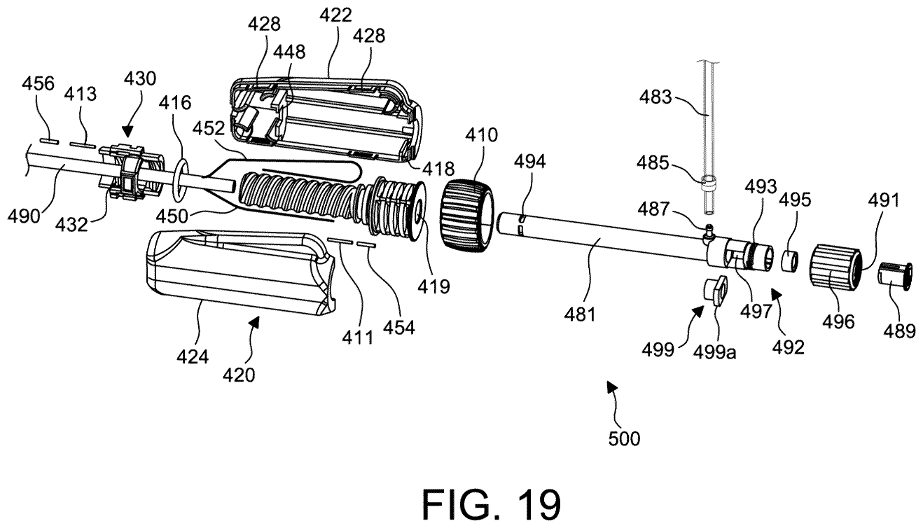

As illustrated in FIG. 19, the proximal assembly 492 includes a nut 496 having internal threads 491. The nut 496 is configured to translate over a proximal portion of the proximal assembly 492. Internal threads 491 mate with external threads 493 on the proximal assembly. Rotation of the nut 496 allows for opening and closing of a valve 495 located within a proximal channel 497 of the proximal assembly 492. The valve 495 allows for introduction of a medical device or fluid into the sheath 490 from the aperture 498 when in an opened configuration.

The valve 495 is opened and closed by rotation of the nut 496, which translates a compressor 499 that is located proximally of the valve 495 in the proximal channel 497. The compressor 499 includes a flange 499a that is held within recess or engaged with a shoulder within the interior of the nut 496. As such, as the nut 495 is rotated in a first direction, the compressor 499 is translated distally and compresses the valve 495. This causes the valve 495 to close. As the nut 496 is rotated in a second direction, the compressor 499 is translated proximally and allows the valve 495 to decompress. Thus, allowing the valve 495 to open. In another embodiment, the compressor 499 is not held by the nut 496. Rather, the compressor 499 freely moves within the nut 496. When the nut 496 is rotated in a first direction, the compressor 499 is pushed against the valve 495 compressing the valve 495. When the nut 496 is rotated in a second direction, space is created between the compressor 499 and the nut 496 providing the compressor 499 room to move. This allows the valve 495 to expand, thus opening, and push the compressor 499 proximally. A snap 489 is positioned proximally of the compressor 499 in the proximal channel 497 of the proximal assembly 492. The snap 489 locks into the proximal channel 497 and defines the aperture 498.

As illustrated in FIG. 19, the proximal assembly 492 includes an elongated cylindrical body 481 having one or more recess 494 at a distal portion thereof. The one or more recess 494 is configured mate with a shoulder 448 extending from an inner surface of a right handle housing 422. The shoulder 448 is c-shaped and partially surrounds the elongated cylindrical body 481. The elongated cylindrical body 481 is configured to support a screw 414 and ensures the longitudinal axis of the screw 414 remains centered along the longitudinal axis of the handle 400.

The proximal assembly 492 also includes a port 487. Port 487 connects with flexible tubing 483 and allows an additional channel for the introduction of fluids into or out of the sheath 490. A tube clamp 485 ensures the flexible tubing 483 remains secured to port 487 when connected thereto.

As shown in FIG. 19, the handle housing 420 includes a right handle housing 422 and a left handle housing 424 which are configured to join together to encase components within the handle housing 420. The right handle housing 422 and the left handle housing 424 also include connecting elements 428, such as snap fit features, to allow the right handle housing 422 and the left handle housing 424 to be joined together.

The handle 500 comprises a knob 410 that is rotatably coupled to a proximal end 423 of the handle housing 420. The knob 410 is rotatable about the longitudinal axis of the handle 500 and rotates with respect to handle housing 420. In operation, the rotation of the knob 410 in a first rotational direction allows the user to steer or deflect the sheath 490 in a first direction, whereas the rotation of the knob 410 in a second rotational direction allows the user to steer or deflect the sheath 490 in a second direction.

The rotation of the knob 410 is converted into a deflection of the sheath 490 via a slide assembly 430 and screw 414. Generally, knob 410 is mounted onto a proximal end of screw 414, such that screw 414 rotates as the knob 410 is rotated. In one embodiment, knob 410 can be formed of a softer material that is attached to a portion of the screw 414 that extends out of housing 420. The knob 410 may include a plurality of raised portions 412 that aid a user in gripping the knob 410. An O-ring 416 sits in a channel 417 on the screw 414 and interacts with a projection 418 extending from an internal surface of the housing 420. The O-ring 416 is configured to dampen vibrations and provide a smooth feel as the knob 410 is rotated.

The screw 414 include a lumen 419 that passes through the longitudinal axis thereof. The elongated cylindrical body 481 is configured to pass through the lumen 419 and support the screw 414. Sheath 490 is received in and affixed to a lumen passing through the proximal assembly 392

The screw 414 is co-operatively engaged with the slide assembly 430 which is housed within the handle housing 420. The screw 414 includes a self-locking thread that communicates with internal threads 432 of the slide assembly 430. As the knob 410 is rotated, the threads of the screw 414 cause translation of the slide assembly 430 via interaction with internal threads 432. In one embodiment, the slide assembly 430 translates proximally as the knob 410 is rotated in clockwise direction and distally when the knob 410 is rotated in a counter-clockwise direction. In another embodiment, the slide assembly 430 translates distally as the knob 410 is rotated in clockwise direction and proximally when the knob 410 is rotated in a counter-clockwise direction.

The threads of the screw 414 can include a dual lead thread with a lead in the range of 0.25″ to 0.50″. In one embodiment, the lead can be 0.30″. In another embodiment, the lead can be 0.45″. The pitch diameter of the threads of the screw 414 can be in the range of 0.5″ to 0.7″. In one embodiment, the pitch diameter is 0.555″. The threads may include an angle in relation to the surface of the screw 414 in the range of 80 degrees to 130 degrees. In one embodiment, the angle is 80 degrees. In another embodiment, the angle is 120 degrees. The threads of the screw 314 can be configured such that between 2 complete turns of the knob 410 and 4 complete turns of the knob 410 translates the slide assembly 430 from a most proximal position to a most distal position. In one embodiment, the threads are configured such that 2.2 turns of the knob 410 can move the slide assembly 430 from a most proximal position to the most distal position or vice versa. In another embodiment, the threads are configured such that 3.3 turns of the knob 410 can move the slide assembly 430 from a most proximal position to the most distal position or vice versa. The threads of the screw 414 are designed to be self-locking. A coefficient of friction greater than 0.11 ensures that the threads are self-locking. In one embodiment, the coefficient of friction is 0.13.

In use, rotation of knob 410 causes a corresponding linear translation of the slide assembly 430 within the housing 420. The slide assembly 430 is coupled to at least one control wire associated with a distal portion of sheath 490. The translation of the slide assembly 430 is converted into a tensioning of the at least one control wire coupled to the slide assembly 430 and thereby resulting in a deflection of the sheath 490.

More specifically, slide assembly 430 is coupled to respective proximal ends of a pair of control wires that extend substantially along the length of the sheath 490, for example a first control wire 450 and a second control wire 452. A distal end (not shown) of each of the control wires 450, 452 is coupled to a distal portion of the sheath 490. The rotation of the knob 410 in one direction causes the slide assembly 430 to translate proximally within the housing 420 pulling one of the control wires (such as first control wire 450) to deflect the sheath 490 in a first direction, whereas the rotation of the knob 410 in an opposing direction causes the slide assembly 430 to translate distally within the housing 420 pulling the other of the control wires (such as second control wire 452) to deflect the sheath 490 in a second direction. While slide assembly 430 applies tension to the first control wire 450, the second control wire 452 is free to move unencumbered through the slide assembly 330. Similarly, as the slide assembly 430 applies tension to the second control wire 452, the first control wire 450 is free to move unencumbered through the slide assembly 430. As such, tension is only applied to one of the first control wire 450 or the second control wire 452 at any time.

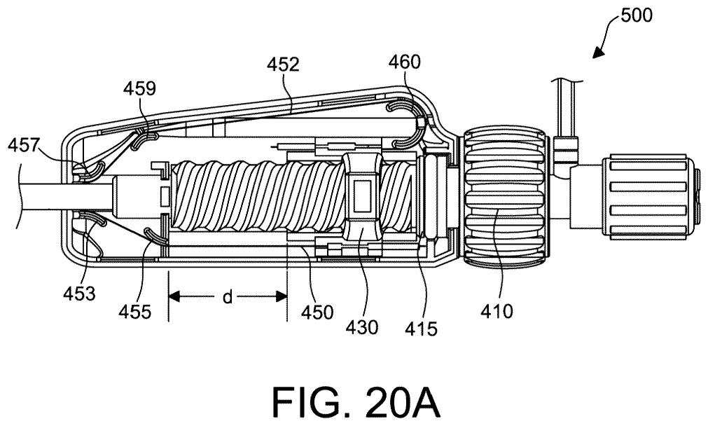

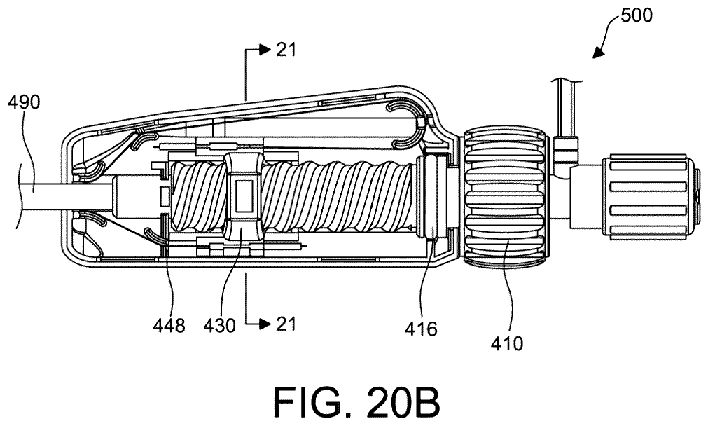

The amount of curvature imparted on the sheath 490 is limited by how far the slide assembly 430 translates within the handle 500. FIG. 20A shows the slide assembly 430 in a most proximal position. The slide assembly 430 is limited proximally by interaction with flange 415 forming a portion of channel 417 to hold O-ring 416. FIG. 20B shows the slide assembly 430 in a most distal position. The slide assembly 430 is limited distally by interaction with shoulder 448. The slide assembly 430 is configured to translate a within the handle 500 a distance d in the range of 0.95″ to 1.5″. In one embodiment, the slide assembly 430 is configured to translate a distance d of 0.99″. In another embodiment, the slide assembly 430 is configured to translate a distance d of 1.27″.

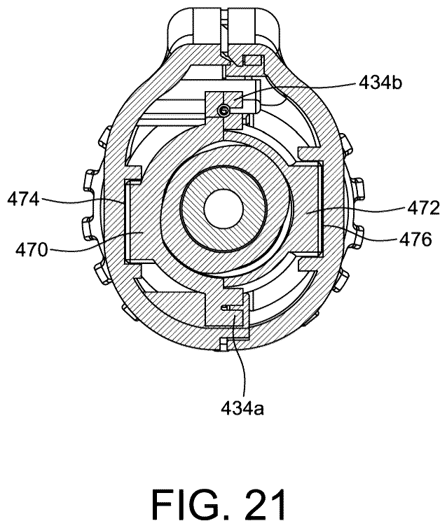

FIG. 21 is a cross-sectional view through the slide assembly 430 along line 21-21 in FIG. 20B. As shown, the slide assembly 430 includes a first raised portion 470 and a second raised portion 472 that is configured to translate within a first guide 474 and a second guide 476 respectively. The first guide 474 and the second guide 476 extend along the inner portion of right handle housing 422 and the left handle housing 424. The first guide 474 and the second guide 476 have a shape that corresponds to the shape of the first raised portion 470 and the second raised portion 472. In one embodiment, the first raised portion 470 and the second raised portion 472 are substantially rectangular as shown FIGS. 20A and 20B. The interaction of the raised portions 470, 472 and the guides 474, 476 allows the slide assembly 430 to translate along the handle 500 without rotating wile knob 410 is rotated. As illustrated in FIG. 21, the first guide 474 and first raised portion 470 are positioned off center from a longitudinal axis passing through the screw 414 while second guide 476 and second raised portion 472 are aligned with the longitudinal axis. This configuration ensures that slide assembly 430 cannot be improperly oriented during assembly.

The slide assembly 430 includes a first wire retainer 434a located at the bottom of the slide assembly 430 and a second wire retainer 434b located at a top of the slide assembly 430. The first wire retainer 434a and the second wire retainer 434b are positioned on opposite sides of the slide assembly 430. In one example as shown in FIGS. 20A and 20B, in order to allow the slide assembly 430 to separately impart a pulling force on each of the two control wires, one of the two control wires (such as first control wire 450) is coupled to the first wire retainer 434a of the slide assembly 430 whereas the other of the control wires (such as second control wire 452) is coupled to the second wire retainer 434b of the slide assembly 430.

The first control wire 450 exits the sheath 490 within the housing 420 and contacts a first control wire first guide 453 and a first control wire second guide 455 before coupling with the first wire retainer 434a. First control wire first guide 453 and first control wire second guide 455 can include a static or movable bearing, pulley, post, pin, curved wall, or channel to position the first control wire 450 in a desired orientation. First control wire first guide 453 and first control wire second guide 455 position the first wire 450 such that the first wire 450 remains substantially parallel to a longitudinal axis of the screw 414 proximal of the first control wire second guide 455. The first control wire 450 is coupled to the first wire retainer 434a such that proximal movement of the slide assembly 430 causes tension on the first control wire 450, allowing deflection of the sheath 490 in a first direction.

The second control wire 452 exists the sheath 490 within the housing 420 and contacts a second control wire first guide 457, a second control wire second guide 459, and a reversing element 460 before coupling with the second wire retainer 434b. The second control wire first guide 457, second control wire second guide 459, and the reversing element 460 may include a static or movable bearing, pulley, post, pin, curved guide, wall, or channel through or around which the second control wire 452 passes to position the second control wire 452 in a desired orientation. Second control wire first guide 457, second control wire second guide 459, and reversing element 460 position the second wire 452 such that the second wire 452 remains substantially parallel to a longitudinal axis of the screw 414 distal of the reversing element 460.

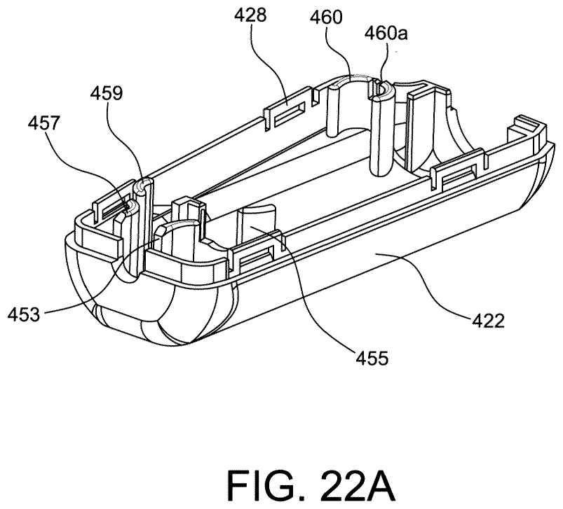

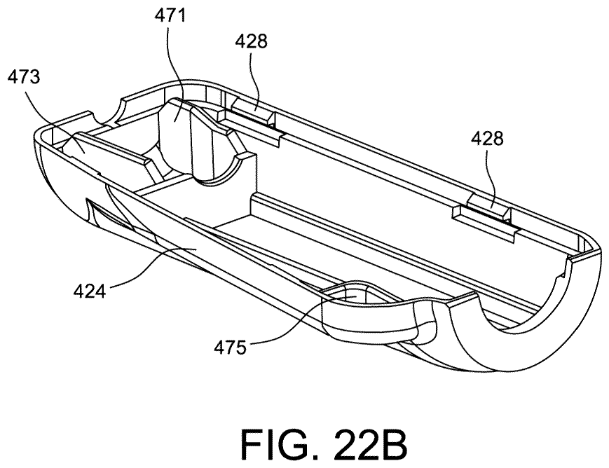

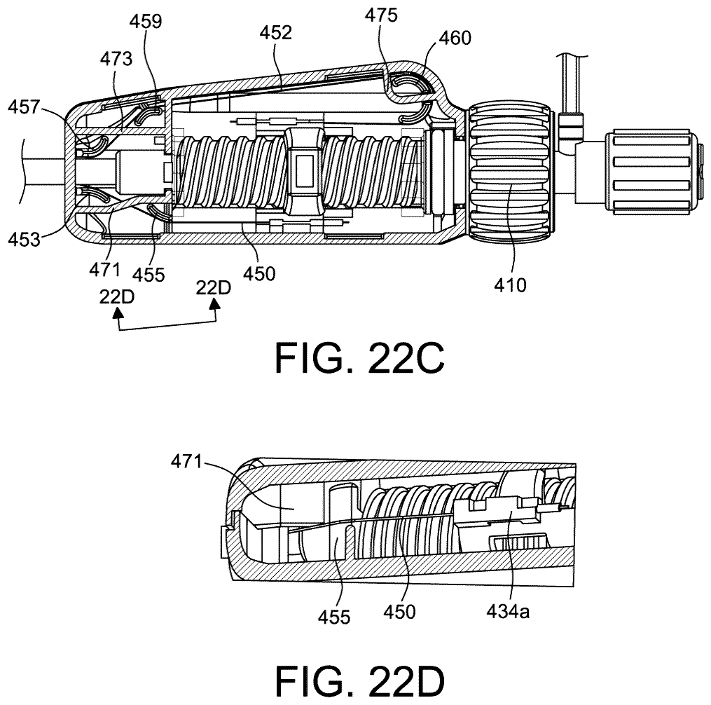

In one embodiment, the first control wire first guide 453, the first control wire second guide 455, the second control wire first guide 457, second control wire second guide 459, and the reversing element 460 each extend from an inner surface of the right handle housing 422 as seen in FIG. 22A.

As shown in FIG. 22B, the left handle housing 424 includes a first guide rib 471, a second guide rib 473, and a third guide rib 475 extending from an inner surface thereof. The first guide rib 471, second guide rib 473, and third guide rib 475 are configured such that upon assembly of the right handle housing 422 and the left handle housing 424, the first guide rib 471, second guide rib 473, and third guide rib 475 constrain the first control wire 450 and the second control wires 452 in a plane passing through the longitudinal axis of the screw 414.

FIG. 22C is a transverse partial cross-sectional view of the steerable handle of FIG. 18 showing the location of the first guide rib 471, second guide rib 473, and third guide rib 475 in relation to the first control wire first guide 453, the first control wire second guide 455, the second control wire first guide 457, second control wire second guide 459, and the reversing element 460. As seen in FIG. 22C, the first guide rib 471 passes between a portion of the first control wire first guide 453 and the first control wire second guide 455 to interact with the first control wire 450. The second guide rib 473 passes between a portion of the second control wire first guide 457 and the second control wire second guide 459 to interact with the second control wire 452. The third guide rib 475 passes through a slot 460a in the reversing element 460 and passes around an end of the reversing element 460 to interact with the second control wire 452.

FIG. 22D is a partial cutaway side view showing the interaction of the first guide rib 471 of the left handle housing with first control wire 450 as viewed from line 22D-22D in FIG. 22C. As shown, the first guide rip 471 interacts with the first control wire 450 to maintain the first control wire 450 in a plane passing through the longitudinal axis of the screw 414.

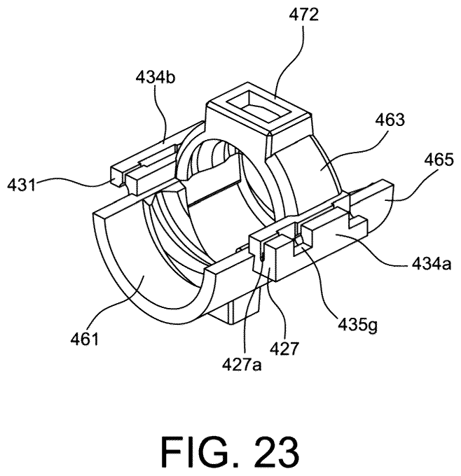

As shown in FIG. 23, the slide assembly 430 includes three sections forming a generally ring-shape. The first section 461, second section 463, and third section 465 are each approximately half of a circle arranged such that the ends connect to form a complete ring. The first section 461 is separated from the third section 465 by the second section 463. The first section 461 and the third section 465 are each located on a first half of the slide assembly 430. The second section 463 forms the second half of the slide assembly 430. The first section 461, second section 463, and third section 465 each include an inner surface and an outer surface. The inner surface of each section includes internal threads that interact with the threads of the screw 414. The outer surface of the first section 461 includes the first raised portion 470. The second raised portion 472 is located on the outer surface of the second section 463. In one embodiment, the third second 465 includes an additional raised portion.

The first wire retainer 434a and the second wire retainer 434b span partially across an outer surface of the first section 461, the second section 463, and the third section 465. The first wire retainer 434a and the second wire retainer 434b are configured to receive and secure the distal ends of first and second control wires 450, 452 respectively.

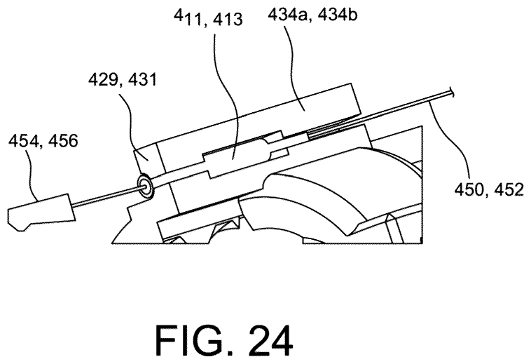

As illustrated in FIGS. 23 and 24, the first wire retainer 434a includes a distal face 427 and a proximal face 429. The distal face 427 includes a channel 427a for receiving a portion of the first control wire 450. The proximal face 429 includes a channel 429a for receiving a length of hypotube 411. The hypotube 411 is configured to provide rigidity to the first wire retainer 434a. The hypotube 411 extends partially through the first wire retainer 434a and rests against a distal surface of a gap 435g.

The hypotube 411 in combination with the distal face channel 427a forms a passage that is configured to receive and secure the first control wire 450. The first control wire 450 passes though the distal face channel 427a and the hypotube, and a first control wire crimp 454 is attached to the distal end of the first control wire 450. The first control wire crimp 454 rests against the hypotube 411 which prevents the first control wire 450 from being pulled from the first wire retainer 434a. As the slide assembly 430 translates proximally within the handle 500, the first control wire crimp 454 presses against the hypotube 411 and tension is applied to the first control wire 450.

The second wire retainer 434b includes a distal face 431 and a proximal face 433. The proximal face 433 includes a channel 433a for receiving a portion of the second control wire 452. The distal face 431 includes a channel 431a for receiving a length of hypotube 413. The hypotube 413 is configured to provide rigidity to the second wire retainer 434b. The hypotube 413 extends partially through the second wire retainer 434b and rests against a proximal surface of a gap 437g.

The hypotube 413 in combination with the proximal face channel 433a forms a passage that is configured to receive and secure the second control wire 452. The second control wire 452 passes though the proximal face channel 433a and the hypotube 413, and a second control wire crimp 456 is attached to the distal end of the first control wire 450. The second control wire crimp 456 rests against the hypotube 411 which prevents the second control wire 452 from being pulled from the second wire retainer 434b. As the slide assembly 430 translates distally within the handle 500, the second control wire crimp 456 presses against the hypotube 411 and tension is applied to the second control wire 452.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C or variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A handle for a steerable medical device, the medical device including a first control wire and a second control wire, a distal end of each of the control wires being coupled to the medical device at a distal region thereof, the handle comprising:
a housing;
a slide assembly positioned within the housing and operable to translate linearly therein, the slide assembly comprising a first wire retainer and a second wire retainer; and
a knob rotatably coupled to the housing, for linearly translating the slide assembly, thereby enabling the slide assembly to manipulate the first control wire and the second control wire to effect a change in a deflection of the medical device;
wherein the slide assembly comprises a ring-shape formed from a first section, a second section, and a third section, wherein the first section and the third section are separated by the second section.

2. The handle of claim 1, wherein first section and the third section form a first half of the ring-shape and the second section forms a second half of the ring-shape.

3. The handle of claim 1, further comprising a screw that includes a thread, wherein rotation of the knob causes rotation of the thread.

4. The handle of claim 3, wherein the first section, the second section, and the third section comprise a threaded inner surface that corresponds to the thread of the screw.

5. The handle of claim 1, wherein the first wire retainer includes a distal face having a channel, a proximal face having channel, and a first wire retainer block located between the distal face and the proximal face.

6. The handle of claim 5, further comprising a second wire retainer, wherein the second wire retainer includes a distal face having a channel, a proximal face having channel, and a second wire retainer block located between the distal face and the proximal face.

7. The handle of claim 6, wherein the first wire retainer block has a proximal end, a distal end, and an overhang extending between the first wire retainer block proximal end and the first wire retainer block distal end, and the second wire retainer block has a proximal end, a distal end, and an overhang extending between the second wire retainer block proximal end and the second wire retainer block distal end.

8. The handle of claim 1, wherein the first wire retainer partially extends across an outer surface of the first section, the second section, and the third section.

9. The handle of claim 1, wherein the first wire retainer includes a hypotube.

10. The handle of claim 3, wherein the knob is integral with the screw.

11. The handle of claim 1, wherein the knob is located at a proximal end of the housing.

12. The handle of claim 1, wherein the first section includes an outer surface having a first raised portion configured to translate within a first guide that extends along an inner portion of the housing.

13. The handle of claim 12, wherein the second section includes an outer surface having a second raised portion configured to translate within a second guide that extends along an inner portion of the housing.

14. The handle of claim 13, wherein the first raised portion and the first guide are positioned off center from a longitudinal axis passing through the handle.

15. A slide assembly for a steerable medical device, the medical device including at least one control wire configured to impart a change in shape of the steerable medical device, the slide assembly comprising:
a first section having a threaded inner surface and an outer surface, the outer surface including a first raised portion;
a second section having a threaded inner surface and an outer surface, the outer surface including a second raised portion;
a first wire retainer extending partially along the first section outer surface and the second section outer surface; and
a second wire retainer extending partially along the first section outer surface and the second section outer surface.

16. The slide assembly of claim 15, wherein the first wire retainer is located on an opposite side of the outer surface than the second wire retainer.

17. The slide assembly of claim 15, further comprising a third section, wherein the first section and the third section are separated by the second section.

18. The slide assembly of claim 17, wherein the first section, the second section, and the third section form a generally ring-shape.

19. A slide assembly for a steerable medical device, the medical device including at least one control wire configured to impart a change in shape of the steerable medical device, the slide assembly comprising:
a first section having a threaded inner surface and an outer surface;
a second section having a threaded inner surface and an outer surface;
a third section having a threaded inner surface and an outer surface; and
a first wire retainer extending partially along the first section outer surface, the second section outer surface, and the third section outer surface;
wherein the first section, the second section, and the third section form a generally ring-shape.

20. The slide assembly of claim 19, further comprising a second wire retainer extending partially along the first section outer surface, the second section outer surface, and the third section outer surface.
